SEQUENCE LISTING (8 sequences)

SEQ ID NO: 1 - DNA, 25 bp, artificial sequence
  siRNA
cguuaaucgcguauaauacgcguat

SEQ ID NO: 2 - DNA, 25 bp, artificial sequence
  siRNA
gcauuugaucagaccaaacagugct

SEQ ID NO: 3 - DNA, 25 bp, artificial sequence
  siRNA
ggccgucuaugaggcagugcugcgg

SEQ ID NO: 4 - DNA, 25 bp, artificial sequence
  siRNA
cgaagucaacaauaccauucuucac

SEQ ID NO: 5 - DNA, 24 bp, artificial sequence
  siRNA, nucleotides 1-20 are methylated
gcauuugaucagaccaaacagugc

SEQ ID NO: 6 - protein, 22 aa, artificial sequence
  protamine fragment with ScFv
RSQSRSRYYRQRQRSRRRRRRS

SEQ ID NO: 7 - DNA, 20 bp, artificial sequence
  DNA version of gRNA
gccgctcacacaggcgctgc

SEQ ID NO: 8 - DNA, 19 bp, artificial sequence
  DNA version of guide RNA
gcggaagaaccctgctgtg

(19) United States
(12) Patent Application Publication
(10) Pub. No.: US 2022/0372490 A1
(43) Pub. Date: Nov. 24, 2022

(54) REACTIVATING P53 MUTANTS FOR CANCER TREATMENT BY TARGETING PROLIDASE (PEPD)

(71) Applicant: Health Research, Inc., Butfalo, NY (US)

(72) Inventors: Yuesheng ZHANG, Orchard Park, NY (US); Yun LI, Orchard Park, NY (US); Lu YANG, Buffalo, NY (US)

(21) Appl. No.: 17/638,518

(22) PCT Filed: Aug. 28, 2020
(86) PCT No.: PCT/US2020/048547

§ 371 Date: Feb. 25, 2022

Related U.S. Application Data
(60) Provisional application No. 62/893,367, filed on Aug. 29, 2019.

(51) Int. Cl. C12N 15/113; A61P 35/00

(52) U.S. Cl. CPC C12N 15/1137; A61P 35/00; C12N 2310/14

(57) ABSTRACT

Provided are methods for prophylaxis or therapy of cancer. The methods are directed to cancers that are characterized by expression of a mutant p53. The mutant p53 may be a loss of function p53 mutant, dominant negative p53 mutant, or a gain of function p53 mutant. The method comprises delivering to cancer cells an agent that can inhibit expression of prolidase (PEPD) or disrupts the association of mutant p53 with PEPD.

### CROSS-REFERENCE TO RELATED APPLICATIONS

This application claims priority to U.S. provisional patent application No. 62/893,367, filed Aug. 29, 2019, the entire disclosure of which is incorporated herein by reference.

### STATEMENT REGARDING FEDERALLY SPONSORED RESEARCH OR DEVELOPMENT

This invention was made with government support under grant nos. CA215093 and CA164574 awarded by the National Institutes of Health. The government has certain rights in the invention.

### BACKGROUND

p53 tumor suppressor plays a crucial role in suppressing cancer development and progression but is commonly mutated in all types of human cancer. There are numerous p53 mutants, the vast majority of which are a single amino acid change in the DNA binding domain. Mutated p53 loses its tumor suppressor activity or gains oncogenic activity. There is an ongoing and unmet need for compositions and methods for treating cancers that express such p53 mutants. The disclosure is pertinent to this need.

### SUMMARY

The present disclosure provides compositions and methods for prophylaxis and/or therapy for cancers. The methods generally involve targeting peptidase D (PEPD), also known as prolidase, in cancer cells that express mutant forms of p53 that promote cancer cell proliferation.

In arriving at the presently provided disclosure, whether PEPD binds and regulates p53 mutants was analyzed, because the p53 domain (PRD) responsible for PEPD binding is intact in almost all cancer-associated p53 mutants. We recently found that PEPD binds and suppresses wild-type (normal) p53 and disrupting the association by targeting PEPD frees and activates p53, causing cell death. However, a main concern was that if PEPD does bind to p53 mutants, targeting PEPD might free p53 mutants and unleash their dominant negative effects or oncogenic effects, i.e., promoting cancer cell growth and proliferation.

The disclosure includes evaluation of common cancer-associated p53 mutants, including R175H, R248Q, R273H, R280K and E285A. These mutants are widely known to have lost tumor suppressor function and gained dominant negative function or oncogenic function. However, to our surprise, rather than freeing the p53 mutants to promote cell survival and growth, PEPD knockdown (KD) by siRNA causes death of cancer cells expressing p53 mutants and evokes molecular changes indicative of WT-p53 activity. This phenomenon is not restricted to a specific mutant, with a certain exception described below. Cell death caused by PEPD loss is clearly caused by the p53 mutants, because knocking out the mutant renders cells insensitive to PEPD suppression. These findings challenge the current understanding about the biology of p53 mutants, reveal a critical regulatory mechanism of p53 mutants, and therefore provide a novel strategy to reactivate p53 mutants, a strategy that may be applicable to most if not all p53 mutants that may or may not have an oncogenic effect. In embodiments, the disclosure thus comprises inhibiting expression of PEPD in cancer cells that harbor p53 mutants. The disclosure is illustrated in non-limiting embodiments using RNA inhibition and PEPD knockouts. Results include a demonstration of the in vivo effect of PEPD knockdown on the growth of and expression of key proteins in isogenic tumors with or without expression of a p53 mutant in a relevant animal model. Accordingly, in embodiments, the disclosure comprises introducing into cancer cells that comprise p53 mutants which may be loss of function p53 mutants, dominant negative p53 mutants, and gain of function p53 mutants.

### BRIEF DESCRIPTION OF THE FIGURES

FIG. 1. PEPD knockdown reactivates a p53 mutant. A, cells were treated with siRNA (10 nM) for 48 h, from which whole cell lysates were prepared and analyzed by Western blotting (WB). Glyceraldehyde 3-phosphate dehydrogenase (GAPDH) is a loading control. B, cells were treated with siRNA (10 nM) for 72 h and then analyzed by trypan blue viability assay (mean±SD, n=3). *P<0.0001.

FIG. 2. PEPD KD by siRNA may induce transcription-independent tumor suppressor activity of p53^{E285A} mutant. A. WB analysis of p53 relocation in cells after siRNA (10 nM) treatment for 72 h. Lamin A, α-tubulin and voltage-dependent anion channel (VDAC) were measured to rule out cross contamination of the subcellular fractions or as loading controls. B, WB analysis of p53 binding to cyclophilin D (CYPD) in mitochondria after cell treatment with siRNA (10 nM) for 72 h. C, flow cytometry analysis of mitochondria membrane potential (MMP) after cell treatment with siRNA (10 nM) for 72 h. Error bars are means±SD, calculated from 3 experiments.

FIG. 3. PEPD KD induces phosphorylation and transcriptional activity of p53 mutant in BT-474 cells. A, reporter activity in cells after plasmid transfection for 24 h and then treatment with siRNA (10 nM) for 48 h. PG13-luc is a p53^{WT} reporter which contains multiple copies of p53^{WT} binding site, whereas MG15-luc contains multiple copies of mutated p53 binding site and does not respond to p53^{WT}. Cells were transfected with either PG13-luc or MG15-luc along with pRL-TK for control of transfection efficiency. B & C, phostag-WB and WB analyses of phosphorylation of p53^{E285A} in cells treated with siRNA (10 nM) for 72 h. D, WB analysis and phostag-WB analysis of nuclear fraction and cytosol of cells treated with siRNA (10 nM) for 72 h. Lamin A and GAPDH were measured as loading controls.

FIG. 4. PEPD binding to p53 mutant in BT-474 cells and the effect of PEPD KD. A-D, Immunoprecipitation (IP)-WB analysis of percentages of cellular p53^{E285A} that binds to PEPD and cellular PEPD that binds to p53^{E285A}. IB bands were quantified by ImageJ. Error bars are mean±SD (n=3). E, WB analysis of PEPD and p53^{E285A} and IP-WB analyses of PEPD-free p53^{E285A} and phosphor-p53^{E285A} in nuclear fraction of cells treated with siRNA for 72 h. Lamin A was measured as a loading control.

FIG. 5. PEPD KO by CRISP-Cas9 kills human cancer cells in a p53-dependent manner. Cells were treated with control or PEPD-targeting CRISPR-Cas9 for 72 h, showing representative microscopic images (bar: 100 μm).

MDA-MB-231 (p53R280K) is a human breast cancer cell line. MDA-MB-231 (p53^{-/-}) was generated from MDA-MB-231 (p53^{R280K}) by CRISPR-Cas9 gene editing.

FIG. 6. Binding of PEPD to p53^{WT} and Mutants. A. Direct Binding of PEPD to wild type p53 (p53^{WT}) and mutants, measured by enzyme-linked immunosorbent assay (ELISA). B. WB analysis of whole cell lysates of untreated cells. GAPDH is a loading control. C, D. All PEPD or p53 in a sample was pulled down by IP. An isotype-specific IgG was used as a control. Percentage of p53 and PEPD not bound to each other were calculated by measuring p53 and PEPD in the supernatants with ELISA. The precipitates and inputs were analyzed by WB (FIG. 14A, 14B), and percentages of p53 and PEPD bound to each other were calculated by comparing their band intensities with that of the input, measured by ImageJ. E. PEPD and p53 levels in whole cell lysates, measured by ELISA. Mean±SD (n=3) in (A) and (C-E). See also FIGS. 13 and 14.

FIG. 7. The Effect of PEPD KO on Cell Survival and Levels of Key Proteins, and the Countereffect of PEPDG^{27SD}. A. Viability of cells treated with siRNA (10 nM) for 72 h, measured by trypan blue assay. B. WB analysis of whole cell lysates. GAPDH is a loading control. Cells were treated with siRNA (10 nM) for 48 h. C. All PEPD in whole cell lysates was pulled down by IP, and the supernatant was analyzed by WB. GAPDH is a loading control. p53 is shown in two exposures for clear display of all the bands. Cells were treated with siRNA (10 nM) for 48 h. Lysates of untreated MDA-MB-231 cells were used in WB as a positive control. D, E. Cell viability measured by trypan blue assay, and WB analysis of whole cell lysates. GAPDH is a loading control. Cells were transfected with an equal amount of plasmid, and 24 h later treated with vehicle or PEPD siRNA (10 nM) for 72 h. Mean±SD (n=3) in (A) and (D). ***P<0.001; ****P<0.0001; ns, not significant. See also FIG. 15.

FIG. 8. The Effect of PEPD KD on p53 Mutants in Isogenic Cells. A. WB analysis of whole cell lysates. GAPDH is a loading control. MDA-MB-231(p53^{KO}) cells were transfected with an equal amount of plasmid (empty vector or a p53 mutant) for 48 h. B, C. Cell viability measured by trypan blue assay, and WB analysis of whole cell lysates. GAPDH is a loading control. MDA-MB-231 (p53^{KO} cells were transfected with a p53 mutant (an equal amount of plasmid for each mutant) and 24 h later treated with siRNA (10 nM) for 96 h. Mean±SD (n=3) in (B). ****P<0.0001.

FIG. 9. The Effect of PEPD KD on Transcription-Independent Tumor Suppressor Activities of p53 Mutants. A. WB analysis of subcellular fractions, using voltage-dependent anion channel (VDAC), GAPDH and lamin B as loading controls. Cells were treated by siRNA (10 nM) for 48 h. B. Mitochondrial membrane potential loss measured by JC-1 fluorescence. Cells were treated by siRNA (10 nM) for 48 h. C. Cells were treated by siRNA (10 nM) for 48 h, from which mitochondria were isolated and subjected to IP. The immunoprecipitate was analyzed by WB. D. Apoptosis measured by TUNEL assay. Cells were treated by siRNA (10 nM) for 72 h (see FIG. 15D). Mean±SD (n=3) in (B) and (D). See also FIG. 15 and FIG. 16.

FIG. 10. The Effect of PEPD KD on Phosphorylation and Transcriptional Activities of p53 Mutants. A. WB analysis of whole cell lysates. Cells were treated by siRNA (10 nM) for 24 h. B. Phostag WB analysis of subcellular fractions. Cells were treated by siRNA (10 nM) for 48 h. C. Luciferase activity measured in whole cell lysates. Cells were transfected with an equal amount of PG13-Luc or MG15-Luc along with pRL-TK and 24 h later treated with siRNA (10 nM) for 48 h. D. Binding of p53 mutants to the p53^{WT} binding sites in the promoters of CDKN1A (encoding p21) and BBC3 (encoding PUMA), measured by ChIP-qPCR assay. Cells were treated by siRNA (10 nM) for 48 h. Mean±SD (n=3) in (C) and (D). See also FIG. 17.

FIG. 11. The Effect of PEPD KD on Refolding of p53 Mutants, and the Role of K373 Acetylation. A. WB analysis of whole cell lysates. GAPDH is a loading control. Cells were treated with siRNA (10 nM) for 48 h. B. WB analysis of subcellular fractions. SKBR3 cells were treated with siRNA (10 nM) for 48 h. C, D. Cell viability measured by trypan blue assay, and WB analysis of whole cell lysates. GAPDH is a loading control. Cells were treated with siRNA (10 nM) with or without C646 (8 μM) for 72 h. C646 is an inhibitor of p300/CBP which acetylates p53. E, F. Cell viability measured by trypan blue assay and WB analysis of whole cell lysates. GAPDH is a loading control. p53^{R175H/K373R} was transfected into MDA-MB-231 (p53^{KO} cells; 24 h later, the cells were treated with siRNA (10 nM) for 96 h. G-K. Whole cell lysates were subjected to IP of p53 using Pab1620, Pab240 or isotype-matched IgG, and WB analysis of the immunoprecipitates. Pab1620 and Pab240 detect the “wild type” and “denatured” conformations of p53, respectively. The relative p53^{R175H} level in (I) was measured by ImageJ. Cells were untreated (G, H) or treated with siRNA (10 nM) for 48 h with or without C646 (8 μM) (I, J). p53^{R175H/L373R} was transfected into MDA-MD-231 (p53^{KO} cells; 24 h later, the cells were treated with siRNA (10 nM) for 48 h (K). L. WB analysis of subcellular fractions. P53^{R175H/K373R} was transfected into MDA-MB-231 (p53^{KO}) cells; 24 h later, the cells were treated with siRNA (10 nM) for 48 h. Lamin B, α-tubulin and VDAC were used as loading controls and to rule out cross-contamination (B, L). CAL-51 whole cell lysates were used as p53^{WT} input for WB. Mean±SD (n=3) in (B, E). *P<0.05; ****P<0.0001. See also FIG. 18.

FIG. 12. Effect of PEPD KD on the Growth of and Expression of Key Proteins in Isogenic Tumors with or without Expressing a p53 Mutant. A-F. Mice bearing orthotopic breast tumors generated from MDA-MB-231-p53^{R280K}, MDA-MB-231-p53^{KO} or MDA-MB-231-p53^{R17514} cells were treated by intratumor injection of siRNA (10 pmol) every 3 days. Arrows indicate treatment start. The experiments ended 24 or 48 h after the last dose. Mean±SEM (n=13-16). ****P<0.0001. G. WB analysis of tumor tissue homogenates. GAPDH is a loading control. Two tumors per group were analyzed. H. Graphic summary of reactivation of p53 mutants by PEPD KD. A, acetylation; P, phosphorylation; U, mono-ubiquitination; p53m, p53 mutants. See also FIG. 19.

FIG. 13. Characterization of PEPD, p53^{WT}, p53 Mutants, and Cell Lines, Related to FIG. 6. A. Sodium dodecyl sulfate polyacrylamide gel electrophoresis of affinity purified recombinant PEPD, p53^{WT}, p53 mutants, and silver staining. B. Sanger sequencing of p53 in cell lines, focusing on changes at amino acids #175, #248, #273, and #280. C. Measurement of PEPD mRNA level in cells by RT-PCR, using GAPDH mRNA as a control.

FIG. 14. Binding of PEPD to p53^{WT} and Its Mutants in Cells, Related to FIG. 6. A. All PEPD in a sample (whole cell lysate, cytosol or nuclear extract) was pulled down by IP using a PEPD antibody in excess. The precipitate and an input were analyzed by WB. The input for p53 WB was 10%, 15% or 20% of each sample used in PEPD IP, with 10% input for whole cell lysates of SKBR3 and MDA-MB-231, 15% input for cytosols and nuclear extracts of SKBR3 and MDA-MB-231, and 20% input for CAL-51 (whole cell lysate, cytosol, and nuclear extract). WB analysis of select supernatants was also performed to confirm that all PEPD was pulled down. Three experiments were carried out to calculate the binding percentages shown in FIG. 6C. B. All p53 in a sample was pulled down by IP using a p53 antibody in excess. The precipitate and an input were analyzed by WB. WB analysis of select supernatants was also performed to confirm that all p53 was pulled down. The input for PEPD WB was 15% of each sample used in p53 IP. WB analysis of select supernatants was also performed to confirm that all p53 was pulled down. Three experiments were carried out to calculate the binding percentages shown in FIG. 6D. C. WB analysis of nuclear extract and cytosol for lamin B and α-tubulin to rule out cross-contamination.

FIG. 15. The Effect of PEPD KD on Levels of p53 and Other Proteins, Cell Cycle Progression, and Apoptosis, Related to FIG. 7 and FIG. 9. A. WB analysis of whole cell lysates for PEPD and other proteins, using GAPDH as a loading control. Cells were treated with siRNA (10 nM) for 48 h. B. Measurement of cell cycle progression by flow cytometry. Cells were treated with siRNA (10 nM) for 48 h. Mean±SD (n=3). **P<0.01; ****P<0.0001. C. WB analysis of whole cell lysates for cleaved caspase 3, using GAPDH as a loading control. Cells were treated with siRNA (10 nM) for 48 h. D. TUNEL fluorescence staining with Fluor-594 and nuclear fluorescence staining with 4′,6-diamidino-2-phenylindole (DAPI). Cells were treated with siRNA (10 nM) for 72 h prior to staining. Scale bar: 100 μm.

FIG. 16. The Role of MDM2 in Mitochondrial Enrichment of p53^{R175H} in SKBR3 Cells, Related to FIG. 9. A. WB analysis of whole cell lysates, cell lysates minus mitochondria, and mitochondria. GAPDH, VDAC and α-tubulin are used as loading controls and to rule out cross-contamination. Cells were treated with scramble siRNA or MDM2 siRNA (10 nM) and 24 h later treated with scramble siRNA or PEPD siRNA (10 nM) for 48 h. B. WB analysis of cell lysates minus mitochondria. GAPDH is a loading control. The same samples were also subjected to ^{p53Rl75H} ir^{m,} and the immunoprecipitate was analyzed by WB.

Cells were treated with siRNA (10 nM) for 48 h. C. Mitochondria samples and nuclear extracts were subjected to IP using a ubiquitin (Ub) antibody, and the immunoprecipitate was analyzed by WB. Cells were treated with siRNA (10 nM) for 48 h, with ubiquitin aldehyde (100 μM) added in the final 4 h, from which mitochondria were isolated.

FIG. 17. The Effect of PEPD KD on Phosphorylation of p53^{WT} and p53 Mutants, Related to FIG. 10. A. WB analysis of whole cell lysates. Cells were treated with siRNA (10 nM) for 48 h. B. Cells were treated by siRNA (10 nM) for 48 h, from which nuclear extracts were prepared. The extracts were subjected to PEPD IP. Both the immunoprecipitate and the supernatant were analyzed by WB and phostag WB. p53 amounts in samples from cells treated with scramble siRNA and PEPD siRNA were equalized for phostag WB.

FIG. 18. The Effect of PEPD KD on Refolding of p53 Mutants, and the Role of K373 Acetylation in Refolding and Reactivation of p53 Mutants, Related to FIG. 11. A. WB analysis of subcellular fractions, including nuclear extract, cytosol and mitochondria. MDA-MB-231 cells were treated with siRNA (10 nM) for 48 h. B. The nuclear extracts of SKBR3 cells and MDA-MB231 cells were subjected to IP with a PEPD antibody in excess to pull down all PEPD, followed by WB analysis of both the precipitate and the supernatant. Cells were treated with siRNA (10 nM) for 48 h. C-F. Cell viability measured by trypan blue assay and WB analysis of whole cell lysates. GAPDH is a loading control. MDA-MB-231cells, MDA-MB-468 cells and HCC70 cells were treated with siRNA (10 nM) with or without C646 (8 μM) for 72 h (C, D). MDA-MB-231 (p53^{KO}) cells were transfected with a p53 mutant, including p53^{R248Q/K373R}, p53^{R273H/K373R}, and p53^{R280K/K373R}, and 24 h later, treated with siRNA (10 nM) for 96 h (E, F). G. Recombinant p53^{WT} and p53^{R175H} were subjected to IP with Pab1620 or Pab240, followed by WB analysis of the precipitate for p53^{WT} and p53^{R175H}. H-K. Whole cell lysates were subjected to IP using Pab1620 or Pab240, followed by WB analysis of the precipitate (H-K) and the supernatant (H). MDA-MB-23 cells, MDA-MB-468 cells, and HCC70 cells were untreated (H) or treated with siRNA (10 nM) for 48 h with or without 8 mM C646 (I, J). MDA-MD-231 (p53^{KO}) cells were transfected with a p53 mutant, including p53^{R248Q/K373R}, p53^{R273H/K373R}, and p53^{R280K/K373R}, and 24 h later treated with siRNA (10 nM) for 48 h (K). Untreated whole cell lysates were used in (H) as a control for WB. L. WB analysis of subcellular fractions. p53^{R248Q/K373R} was transfected into MDA-MB-231 (p53^{KO}) cells; 24 h later, the cells were treated with siRNA (10 nM) for 48 h. Lamin B, α-tubulin and VDAC were used as loading controls and to rule out cross-contamination (A, L). Mean±SD (n=3) in (B, E). *P<0.05; ****P<0.0001.

FIG. 19. Characterization of MDA-MB-231 Cells Stably Expressing p53^{R175H}, Related to FIG. 12. A. WB analysis of whole cell lysates. MDA-MB-231 (p53^{KO}) cells were transfected with p53^{R175H}, and stable clones expressing the mutant were selected by puromycin. SKBR3 cells were used for comparison in WB. GAPDH is a loading control. B. Cell viability measured by trypan blue assay. Cells were treated by siRNA (10 nM) for 72 h. Mean±SD (n=3), P<0.0001.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all polynucleotides disclosed herein, their complementary sequences, and reverse complementary sequences. For any reference to a polynucleotide or amino acid sequence by way of a database entry, the polynucleotide and amino acid sequence presented in the database entry is incorporated herein as it exists on the effective filing date of this application or patent.

In embodiments, the disclosure comprises inhibiting expression of PEPD in cancer cells that express mutant forms of p53 that have lost tumor suppressor function, and/or gained dominant negative function, and/or gained oncogenic function.

Inhibiting expression of PEPD in cancer cells has been described. (Yang et al. Nature Communications, Volume 8, Article number: 2052 (2017)). Briefly, the Yang et al. reference described that PEPD binds and suppresses over half of nuclear and cytoplasmic p53, independent of its enzymatic activity. This reference also showed that PEPD binds to the proline-rich domain (PRD) in p53, which inhibits phosphorylation of nuclear p53 and MDM2-mediated mitochondrial translocation of nuclear and cytoplasmic p53. This reference also showed that eliminating PEPD causes cell death and tumor regression due to p53 activation. The Yang et al., reference used cells that express wild type p53, with the exception of one mutant. However, Yang et al. concluded in that paper that the p53 mutant (an F113C change in p53 found in UM-UC-3 cells) remains functional, and thus despite this mutation, it was believed to retain its wild type function. Thus, the Yang et al. reference did not describe or suggest consequence of separating PEPD from a p53 mutant that is known to have an oncogenic effect.

In contrast, the present disclosure relates to inhibiting interaction between PEPD and mutant forms of p53, which as a consequence, surprisingly causes cancer cell death. Thus, the method of the disclosure is counterintuitive, the expectation being that freeing a mutant p53 that has lost its tumor suppression function and gained oncogenic function from a complex with PEPD should unleash its oncogenic function and accelerate tumor growth. However, data presented in this disclosure unexpectedly demonstrate the opposite effect, namely, disrupting the interaction of PEPD with mutant p53 can be expected to induce cancer cell death.

Accordingly, in embodiments, the disclosure comprises suppressing the interaction of PEPD with p53 in cancer cells that comprise oncogenic mutant p53 proteins, other than mutations which preserve wild type tumor suppressor function. The disclosure accordingly includes the proviso that, in certain non-limiting embodiments, the cancer cells do not comprise mutant p53 in which the only mutation is F113C.

In embodiments, formation of complexes comprising mutant p53 and wild type p53 (e.g., heterodimers) inhibits the function of wild type p53, in the absence of a PEPD targeting agent described herein.

In embodiments, the cancer cells comprise a mutant p53 which, in the absence of a PEPD targeting agent described herein, form a complex with PEPD.

In embodiments, inhibiting formation of a complex of PEPD and a mutant p53 converts the mutant p53 into a p53 that mimics the function of a wild type p53, thereby killing the cancer cells, rather than the otherwise expected outcome of promoting tumor growth. Thus, the disclosure comprises reactivating p53 mutants for cancer treatment by targeting PEPD. In embodiments, subsequent to administering an RNAi agent described herein, PEPD that was initially present in a complex with a mutant p52, is naturally degraded by ordinary cellular protein degradation methods, and due to the presence of the RNAi agent, no replacement PEPD is made, thus enabling the mutant p53 to be freed from PEPD, which gains a wild type confirmation induced by certain posttranslational modifications (lysine 373 acetylation, mono-ubiquitination, and phosphorylation in transactivation domains), and accordingly participate in killing the cancer cells. In embodiments, the disclosure provides for use of an agent that disrupts the association of p53 mutants with PEPD, such as a small molecule or other compound with this function.

In embodiments, the mutant p53 in cancer cells targeted by the agents of this disclosure has lost its normal function as a regulator of transcription, or has lost its normal transcription-independent function, and the abnormal functions and associated mutations are well known in the art.

In embodiments, the cancer cells that are targeted using agents of this disclosure comprise loss of function p53 mutants, dominant negative p53 mutants, and/or gain of function p53 mutants, each of which are also well known in the art.

In embodiments, the cancer cells express a mutant p53, and a wild type p53, and accordingly the individual treated may have tumors that are heterozygous for the p53 allele. In embodiments, the cancer cells may express only mutant p53, and thus the individual treated may be homozygous for the p53 allele.

p53 mutations that promote cancer growth and characterize cancer cells that can be targeted according to this disclosure have been described in detail, for example, in Freed-Pastor and Prives, Genes & Dev (2012) 26: 1268-1286, the disclosure of which is incorporated herein by reference.

The amino acid sequence of p53, and its attendant amino acid locations, are well known in the art and are referred to by amino acid position. In non-limiting examples, the cancer cells express p53 having a mutation that is located at p53 amino acid R175, R248, R273, R280 or E285. Specific and non-limiting examples of mutations at these locations include R175H, R248Q, R273H, R280K and E285A, and additional examples are described below. In embodiments, the p53 mutation is one or any combination of K132Q, A138P, L145R, V147D, P151S, P152L, G154V, T155P, T155N, R156P, V157F, R158L, R158H, A159D, A161T, Y163C, K164N, R175L, C176F, C176Y, C176W, H179Q, R181C, H193L, H193R, H193Y, L194F, R213Q, Y205F, S215I, V216M, Y220C, Y220D, P223L, E224D, I232N, Y234H, Y236C, M237I, N239D, S241F, C242F, C242R, C242S, C242W, R244D, G245R, G245S, R248W, R248L, G245S, G245C, G245D, G245V, M246I, R249S, P250L, I251N, I254D, I255N, G262V, G262D, G266E, G266V, R267L, R267W, V272M, R273C, R273L, V274F, C275G, C275F, C277F, P278A, R280T, R282W, R282G, R283C, R283P, P309S, K320N, Q331R, G334V, G389W.

In embodiments, a p53 mutation in cancer cells targeted by methods of this disclosure alters p53 interaction with one or more other proteins, including but not necessarily limited to NF-y, Spl, Ets-1, VDR, SREBP-2, TopBPi, Pinl, MRE11, PML, p63, or p73. In embodiments, such p53 mutations include those mentioned above, and may further include mutations at p53 position V143, D281, R249, and Y220.

In embodiments, the p53 mutant induces expression of oncogenes, non-limiting examples of which include proliferating cell nuclear antigen (PCNA), EGFR, c-Myc, and mixed lineage leukemia 1 (MLL1).

In embodiments, the p53 mutation in cancer cells when not targeted by PEPD KD as described herein may transactivate other proteins, including but not limited to MYC, CXCL1, PCN, MAP2K3, CCNA, CCNB, CDK1, CDC25C, ASNS, E2F5, MCM6, IGF1R, STMN1, and EGFR, thereby promoting proliferation of cancer cells.

In embodiments, the p53 mutant in cancer cells when not targeted by PEPD KD described herein may up-regulate genes which encode proteins that inhibit apoptosis or promote resistance to chemotherapeutic agents. Such genes include but are not limited to EGR1, ABCB1, IGF2, DUT, BCL2L1, TIMMS° , LGALS3, and NFKB2.

In embodiments, the agent used to inhibit formation of a complex comprising PEPD and mutant p53 is an RNAi agent. In embodiments, the RNAi agent thus has complementarity to an mRNA encoding PEPD. The sequence of human PEPD and mRNA encoding it is known in the art, and the disclosure includes targeting any such mRNA sequence. See, for example, U.S. Pat. No. 10,155,028, from which the description of PEPD is incorporated herein by reference.

Accordingly, in embodiments, expression of PEPD in a cancer cell comprising a mutant p53 as described herein can be inhibited by inhibiting translation of mRNA encoding the PEPD. In embodiments, the mRNA encoding the protein is degraded. In this regard, in non-limiting embodiments, RNA interference (RNAi)-mediated silencing and/or reducing mRNA encoding an PEPD is performed. In embodiments, this is achieved by delivery of any suitable RNAi agent. In embodiments, an siRNA-based approach is used. This can be performed by introducing and/or expressing one or more suitable short hairpin RNAs (shRNA) in the cells. shRNA is an RNA molecule that contains a sense strand, antisense strand, and a short loop sequence between the sense and antisense fragments. shRNA is exported into the cytoplasm where it is processed by dicer into short interfering RNA (siRNA). siRNA are 21-23 nucleotide double-stranded RNA molecules that are recognized by the RNA-induced silencing complex (RISC). Once incorporated into RISC, siRNA facilitate cleavage and degradation of targeted mRNA. Thus, for use in RNAi mediated silencing or downregulation of PEPD expression as described herein, siRNA, shRNA, or miRNA can be used. In alternative embodiments, a functional RNA, such as a ribozyme is used. In embodiments, the ribozyme comprises a hammerhead ribozyme, a hairpin ribozyme, or a Hepatitis Delta Virus ribozyme. In related embodiments, a microRNA (miRNA) adapted to target the relevant mRNA can be used. The term “microRNA” can be used interchangeably with “miR,” or “miRNA” to refer to, for example, an unprocessed or processed RNA transcript from an engineered miRNA gene. The unprocessed miRNA gene transcript is also called a “miRNA precursor,” and typically comprises an RNA transcript of about 70-100 nucleotides in length. The miRNA precursor can be processed by digestion with an RNAse (for example, Dicer, Argonaut, or RNAse III) into an active 19-25 nucleotide RNA molecule. This active 19-25 nucleotide RNA molecule is also called the “processed” miRNA gene transcript or “mature” miRNA. Any of these forms of microRNA can be adapted for use in embodiments of this disclosure. Further, in certain embodiments, the RNAi agent may be provided as a synthetic agent, such as a microRNA mimic, short interfering RNA (siRNA), a RNA interference (RNAi) molecule, double-stranded RNA (dsRNA), short hairpin RNA (shRNA) as described above, primary miRNAs (pri-miRNAs), or small nucleolar RNAs (snoRNAs). Inhibition of the expression of PEPD may therefore be achieved by inhibiting translation, transcription, and/or by mRNA degradation.

In embodiments, the RNAi agent may be modified to improve its efficacy, such as by being resistant to nuclease digestion. In embodiments, the RNAi agent polynucleotides which comprise modified ribonucleotides or deoxyribonucleotide, and thus include RNA/DNA hybrids. In non-limiting examples, modified ribonucleotides may comprise methylations and/or substitutions of the 2′ position of the ribose moiety with an —O-alkyl group containing 1-6 saturated or unsaturated carbon atoms, or with an —O-aryl group having 3-6 carbon atoms, wherein such alkyl or aryl group may be unsubstituted or may be substituted, e.g., with halo, hydroxy, trifluoromethyl, cyano, nitro, acyl, acyloxy, alkoxy, carboxyl, carbalkoxyl, or amino groups; or with a hydroxy, an amino or a halo group. In embodiments modified nucleotides comprise methyl-cytidine and/or pseudo-uridine. The nucleotides may be linked by phosphodiester linkages or by a synthetic linkage, i.e., a linkage other than a phosphodiester linkage. Examples of inter-nucleoside linkages in the polynucleotide agents that can be used in the disclosure include, but are not limited to, phosphodiester, alkylphosphonate, phosphorothioate, phosphorodithioate, phosphate ester, alkylphosphonothioate, phosphoramidate, carbamate, carbonate, morpholino, phosphate triester, acetamidate, carboxymethyl ester, or combinations thereof. In the examples of this disclosure, the following RNAi agents were used:

Nonspecific scrambled control siRNA:
(SEQ ID NO: 1)
rCrGrUrUrArArUrCrGrCrGrUrArUrArArUrArCrGrCrGrUAT
PEPD siRNA #1:
(SEQ ID NO: 2)
rGrCrArUrUrUrGrArUrCrArGrArCrCrArArArCrArGrUrGCT
PEPD siRNA #2:
(SEQ ID NO: 3)
rGrGrCrCrGrUrCrUrArUrGrArGrGrCrArGrUrGrCrUrGrCGG
PEPD siRNA #3:
(SEQ ID NO: 4)
rCrGrArArGrUrCrArArCrArArUrArCrCrArUrUrCrUrUrCAC

In an embodiment, the RNAi agent comprises rGmCrA mUmUmU rGrAmUmCrArG rAmCmCrArArAmCrArG mUrGC T/36-FAM (SEQ ID NO:5), with 2′-O-methylated at all C and U residues of the sense strand. This, or any other RNAi agent, can be provided with further modifications to, for example, enhance delivery. For example, the RNAi agent can be complexed with one or more proteins. In embodiments, the RNAi agent is irreversibly or reversibly attached to a protein. In embodiments, the protein comprises a cancer cell specific binding partner, such as a protein or peptide, including but not necessarily limited to a cancer cell receptor ligand, so that the RNAi agent can be specifically delivered to cancer cells which comprise mutant PEPD. In embodiments, the RNAi agent is used in a complex with a ligand that specifically binds to Her2/neu (human epidermal growth factor receptor type 2), fibroblast growth factor receptor (FGFR), E-cadherin, EMA (epithelial membrane antigen), αvβ6 integrin, EpCAM (epithelial cell adhesion molecule), CEA (carcinoembryonic antigen), FR-α (folate receptor-alpha), or uPAR (urokinase-type plasminogen activator receptor), αvβ3 integrin, bombesin R, carcinoembryonic antigen (CEA), CD13, CD44, C-X-C chemokine receptor-4 (CXCR), carbonic anhydrase-9 (CAIX), emmprin (CD147), endoglin (CD105), epithelial cell adhesion molecule (EpCAM), MET, IFG1R, EphA2, fibroblastic activation protein-alpha (FAP-α), matriptase, mesothelin, MT1-MMP, Muc-1, prostate stem cell antigen (PSCA), prostate-specific membrane antigen (PSMA), Tn antigen, urokinase-type plasminogen activator receptor (uPAR), or VEGFR. In an embodiment, the RNAi agent is provided in a complex with ERBB1, or ERBB2, or a segment thereof. In embodiments, the protein or peptide ligand is present in a fusion protein. In embodiments, the protein or peptide cancer cell specific ligand is provided in a fusion protein that comprises an immunoglobulin, or segment of an immunoglobulin, such as an antibody. Non-limiting examples of such proteins include single-chain variable fragments (scFvs), VHH single chain antibodies, Fabs, single domain antibodies (sdAbs, VHHs), affibodies or darpins. In one embodiment, the ligand and the segment of the immunoglobulin are separated by a linker, such as a flexible GS linker, many suitable examples of which are known in the art. In an embodiment, the RNAi agent is used with a fusion protein of ERBB2 and an scFV-protamine fragment (such as amino acids 8-29). In an embodiment, the protamine fragment comprises or consists of the amino acid sequence RSQSRSRYYRQRQRSRRRRRRS (SEQ ID NO:6). In embodiments, the disclosure comprises a fusion comprising scRv and an arginine polymer (e.g., a nine-mer arginine peptide), such as that described in Lu et al., Biomaterials 2016, 76, 196-207, the description of which is incorporated herein by reference. Such approaches and compositions are known in the art and can be adapted for use with the presently provided RNAi agents in view of the instant disclosure. (See, for example, Song et al., Antibody mediated in vivo delivery of small interfering RNAs via cell-surface receptors. Nature Biotechnology 2005, 23, 709-717; Yao et al., Targeted delivery of PLK1-siRNA by ScFV suppresses HER2+ breast cancer growth and metastasis. Science Translational Medicine 2012, 4, 130ra48; Lu et al., siRNA delivered by EGFR-specific scFV sensitizes EGFR-TKI-resistant human long cancer cells. Biomaterials 2016, 76, 196-207; the disclosures of which are incorporated by reference.)

In embodiments, the disclosure comprises selecting a cancer patient based on having a cancer that expresses a mutant p53 as described herein, and administering to the individual an RNAi-agent that targets the PEPD, thereby disrupting the interaction between PEPD and the mutant p53 protein, and providing the individual with a prophylactic or therapeutic effect against cancer.

In embodiments, the cancer patient has any type of cancer that expresses a mutant p53 protein as described herein. As such, the type of cancer is not particularly limited, and may include but is not necessarily limited to breast cancer, prostate cancer, pancreatic cancer, lung cancer, liver cancer, ovarian cancer, cervical cancer, colon cancer, esophageal cancer, stomach cancer, bladder cancer, brain cancer, testicular cancer, head and neck cancer, melanoma, skin cancer, any sarcoma, including but not limited to fibrosarcoma, angiosarcoma, adenocarcinoma, and rhabdomyosarcoma, and any blood cancer, including all types of leukemia, lymphoma, or myeloma. In embodiments, the cancer comprises triple-negative breast cancer (TNBC). TNBC represents a subset of breast cancer that lacks estrogen receptor (ER), progesterone receptor and HER2 receptor tyrosine kinase. It currently has no targeted therapy, and patients have a poor prognosis.

In embodiments, the RNAi agent can be administered to an individual as a naked polynucleotide, in combination with a delivery reagent, or as a recombinant plasmid or viral vector which comprises and/or expresses the RNAi agent. In one embodiment, the proteins are encoded by a recombinant oncolytic virus, which can specifically target cancer cells, and which may be non-infective to non-cancer cells, and/or are eliminated from non-cancer cells if the oncolytic virus enters the non-cancer cells.

In embodiments, a therapeutically acceptable amount of an RNAi agent is used. A therapeutically effective amount is an amount that can achieve a desired effect, such as reduction in tumor growth rate, inhibition of tumor formation, inhibiting metastasis, or preventing the development of cancer and/or a tumor.

In embodiments, RNAi agents of this disclosure can be combined if desired with a delivery agent. Suitable delivery reagents, in addition to the fusion proteins described above, for administration include but are not limited to Minis Transit TKO lipophilic reagent; lipofectin; lipofectamine; cellfectin; or polycations (e.g., polylysine), liposomes, nanoparticles, or combinations thereof. In embodiments, the RNAi agent may be administered by an intra-tumor injection. In embodiments, nanoparticles or other suitable drug delivery reagents may be used such that the RNAi agent is contained by the nanoparticles. In embodiments, the nanoparticles or other drug delivery reagent may be provided in association with a binding partner that binds specifically to a cancer cell marker. As described above, in embodiments, the binding partner comprises a cancer cell surface receptor ligand. In embodiments, the binding partner comprises an antibody, or antigen binding fragment thereof, which may be provided as a fusion protein with the cancer cell surface receptor ligand. In embodiments, when delivered such that the RNAi and drug delivery reagent are specifically targeted to cancer cells, the administration may comprise any suitable route, including oral, parenteral, subcutaneous, intraperitoneal, intrapulmonary, and intranasal, and intracranial. Parenteral infusions include intramuscular, intravenous, intraarterial, intraperitoneal, and subcutaneous administration. Direct injection into a tumor is also included.

Therapy or inhibition of cancer as described herein may be combined with any other anti-cancer approach, such as surgical interventions and conventional chemotherapeutic agents. In embodiments, cancer treatment according to this disclosure can be combined with administration of one or more immune checkpoint inhibitors. In embodiments, the checkpoint inhibitors comprise an anti-programmed cell death protein 1 (anti-PD-1) checkpoint inhibitor, or an anti-Cytotoxic T-lymphocyte-associated protein 4 (anti-CTLA-4) checkpoint inhibitor. There are numerous such checkpoint inhibitors known in the art. For example, anti-PD-1 agents include Pembrolizumab and Nivolumab. An anti-PD-L1 example is Avelumab. An anti-CTLA-4 example is Ipilimumab. In embodiments, combining an RNAi agent and a chemotherapeutic agent, or an RNAi agent and an immune checkpoint inhibitor, may exhibit a synergistic anti-cancer effect.

The following examples are intended to illustrate certain embodiments of the disclosure, but are not intended to be limiting.

The following experiments were conducted using several human breast cancer cell lines, including 1) MCF-7 (WT-53); 2) CAL-51 (WT-p53); 3) BT-474 (p53^{E285A}); 4) MDA-MB-231 (p53^{R180K}); 5) MDA-MB-231 with p53 knockout (p53^{-/-}), which was generated from MDA-MB-231 (p53^{R280K}) by CRISPR-Cas9; 6) MDA-MB-468 (p53^{R273H}); 7) HCC70 (p53^{R248Q}); 8) SKBR3 (p53^{R175H}). MCF-7 is an estrogen receptor-positive breast cancer cell line. BT-474 and SKBR3 are HER2-positive breast cancer cell lines. The other cell lines are triple negative breast cancer cell lines. The p53 mutant in BT-474 cell is temperature-sensitive, showing mutant activity at 37° C. but reverting to wild type activity at 32° C. All experiments were performed at 37° C.

### EXAMPLE 1

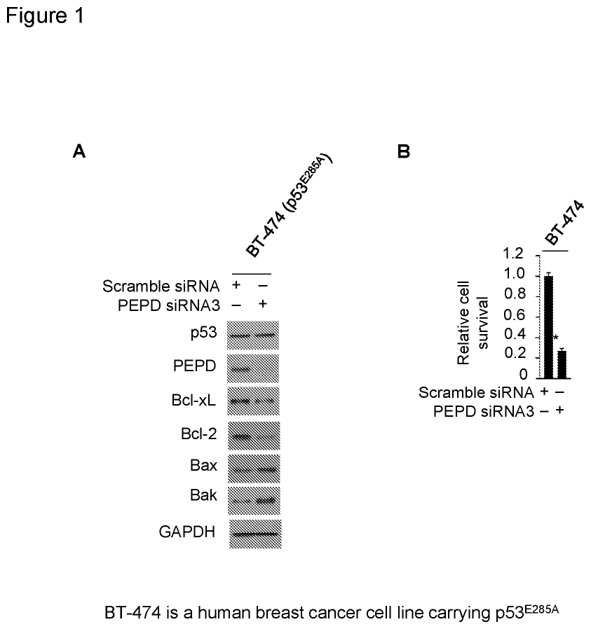

This Example provides results, as shown in FIG. 1, for one hotspot p53 mutant, i.e., p53^{E285A} in BT-474 cells. PEPD KD by siRNA was associated with no change in p53 level but downregulation of Bcl-x1 and Bcl-2 and induction of Bax and Bak (A). The changes in the Bcl-2 family proteins is consistent with marked inhibition of cells growth (B). These results suggested that PEPD KD may reactivate p53^{E285A}.

### EXAMPLE 2

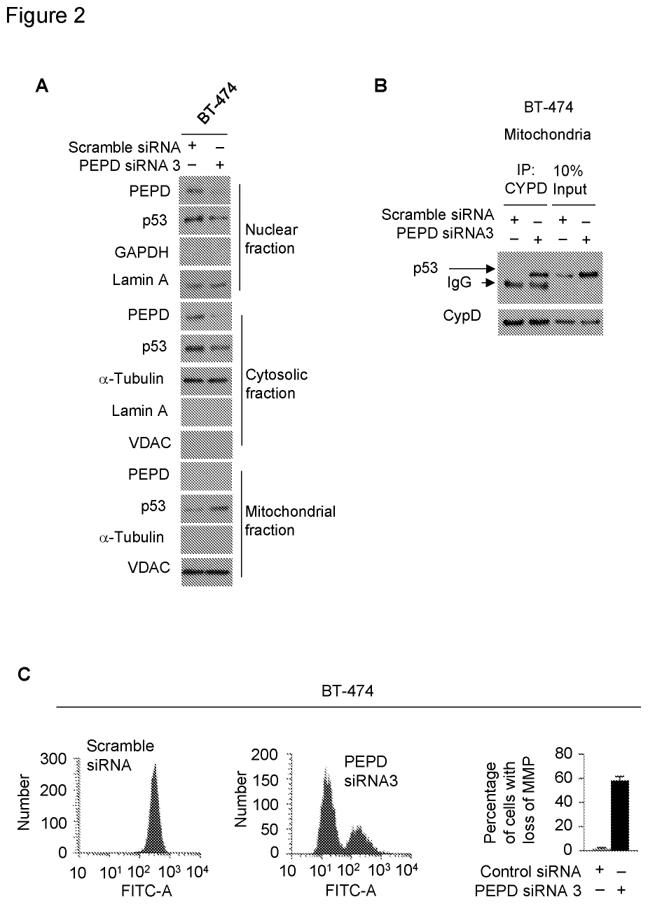

This Example demonstrates that PEPD KD by siRNA may induce transcription-independent tumor suppressor activity of p53^{E285A} mutant. In particular, the data presented in FIG. 2 show that PEPD KD by siRNA induces mitochondria translocation of the p53 mutant from both cytosol and nucleus (A), binding of mitochondria p53 mutant to cyclophilin D (CypD; a key regulator of mitochondria permeability transition pore) (B) and damage of mitochondria (loss of mitochondria membrane potential) (C).

### EXAMPLE 3

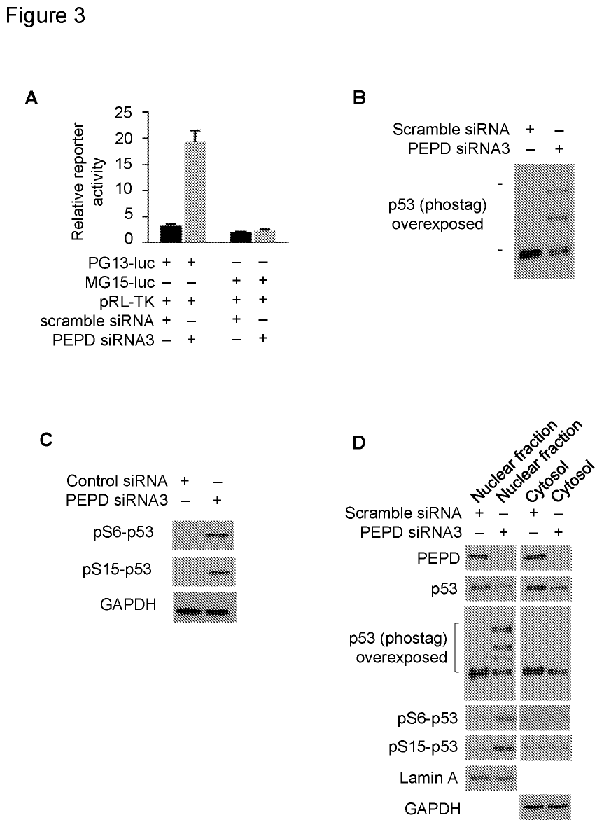

This example suggests reactivation of p53^{E285A} in BT-474 cells. To determine the effect of PEPD KD on transcriptional function of p53^{E285A} BT-474 cells were transfected with an equal amount of p53 reporter PG13-luc which contains multiple copies of p53-binding sequence or MG15-luc which contains multiple copies of mutated p53-binding sequence, along with pRL-TK (Renilla luc) for control of transfection efficiency; 24 h later, cells were treated with control siRNA or PEPD siRNA (10 nM) for 48 h. PG13-luc responded to PEPD KO by increasing luciferase (luc) expression 5.9-fold, whereas luc expression by MG15-luc changed little following PEPD KD (FIG. 3A). This suggests that PEPD KD may markedly increases the transactivation activity of p53^{E285A} which is consistent with modulation of various p53 target proteins by PEPD KD as described before. Moreover, PEPD KD stimulated p53 phosphorylation, as detected by phostag WB analysis (FIG. 3B). Two specific phosphorylation sites in the p53 transactivation domain (serine 6 and serine 15) were measured, and PEPD knockdown caused phosphorylation at both sites (FIG. 3C). By analyzing nuclear fraction and cytosolic fraction separately, we found that PEPD KD-induced p53 phosphorylation occurs in nuclear p53 but not cytosolic p53 (FIG. 3D).

### EXAMPLE 4

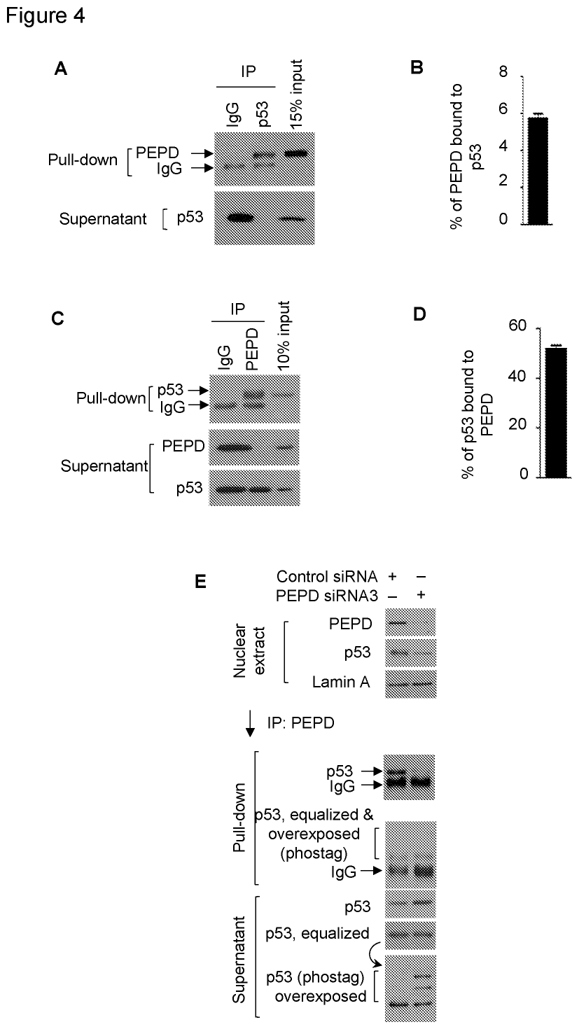

This Example demonstrates PEPD binding to p53 mutant in BT-474 cells. We analyzed p53^{E285A} binding to PEPD in BT-474 cells. To estimate the percentage of cellular PEPD that binds to p53^{E285A} in BT-474 cells, all p53^{E285A} molecules in whole cell lysates were pulled down with a p53 antibody in excess, and the percentage of PEPD molecules that came down with p53^{E285A} was determined by comparing the intensity of the PEPD band with that of the input control (FIG. 4A). We found that about 6% of cellular PEPD molecules are bound to p53^{E285A} in these cells (FIG. 4B). To estimate the percentage of cellular p53^{E285A} molecules that bind to PEPD, all PEPD molecules in cell lysates were pulled down with a PEPD antibody in excess, and the percentage of p53^{E285A} molecules that remained in the supernatant was determined by comparing the intensity of the p53^{E285A} band with that of the input control (FIG. 4C). We found that about 55% of p53^{E285A} molecules in the cell lines are bound to PEPD (FIG. 4D). We also measured p53^{E285A} remaining in the supernatant and found about 45% of the mutant molecules in this fraction (FIG. 4C).

To better understand how PEPD inhibits nuclear p53^{E285A}, we treated BT-474 cells with control or PEPD siRNA, and then analyzed nuclear p53^{E285A}. PEPD knockdown resulted in marked decrease in nuclear p53 level (FIG. 4E). Next, PEPD-bound p53^{E285A} in the nuclear extracts was removed by PEPD pull-down. PEPD KD caused no phosphorylation of p that remained bound to PEPD (pull-down fraction) (FIG. 4E). However, despite p53^{E285A} exit from the nucleus due to PEPD KD, PEPD-free nuclear p53^{E285A} level (supernatant fraction) increased significantly in such cells (FIG. 4E). Moreover, it is the PEPD-free nuclear p whose phosphorylation increased upon PEPD KD (FIG. 4E). Thus, once freed from PEPD, p53^{E285A} isphosphorylated.

### EXAMPLE 5

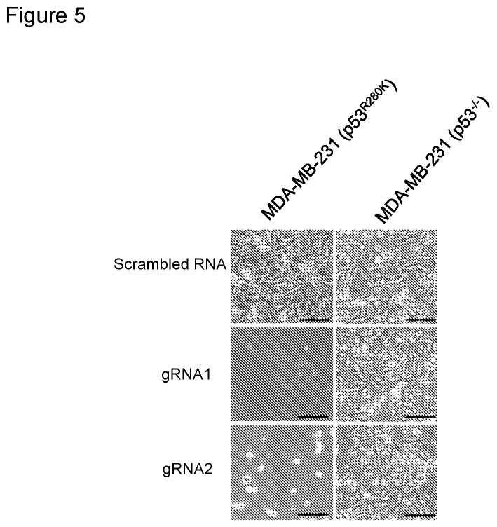

This Example demonstrates that PEPD KO by CRISP-Cas9 kills human cancer cells in a p53-dependent manner. Results are shown in FIG. 5 and demonstrate that killing of cancer cells by PEPD KO requires a p53 mutant. A CRISPR-Cas9 nuclease and gRNA target gene knockout set was purchased from Celltechgen (CTG-CS9O-19761), including the CRISPR Cas9 nuclease expression vector (pST1374-N-NLS-Flag-Cas9-EGFP), PEPD gRNA vector 1 (pGL3-PEPD-sgRNA1), PEPD gRNA vector 2 (pGL3-PEPD-sgRNA2) and the scramble RNA vector. The human PEPD-specific gRNA sequences used for PEPD knockout by CRISPR-Cas9 are as follows as DNA equivalent: gccgctcacacaggcgctgc (gRNAl; SEQ ID NO:7) and gcggaagaaccctgctgtgc (gRNA2; SEQ ID NO:8). MDA-MB-231 (homozygous p53^{R280K}) along with its p53-null isogenic counterpart (MDA-MB-231 p53^{-/-}) were used. They are triple negative breast (TNBC) cells. MDA-MB-231-p53^{-/-} was generated from MDA-MB-231-p53R280K using CRISPR-Cas9 (Mukhopadhyay et al., J Natl Cancer Inst 2019, 111, djz051). Cells were transfected with various plasmid (same amount of plasmid for each cell line) and checked 72 h later.

### EXAMPLE 6

This Example demonstrates binding of PEPD to p53^{WT} and various p53 mutants. In particular, this Example demonstrates PEPD binds to p53 mutants directly and with similar affinity, and that PEPD binds to nearly half of the cytosolic and nuclear contents of each p53 mutant.

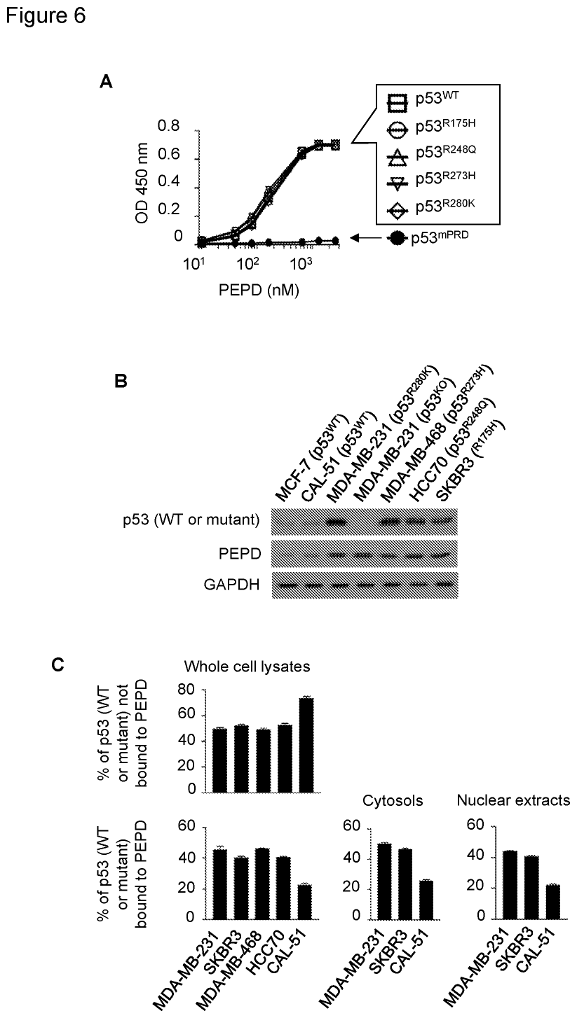

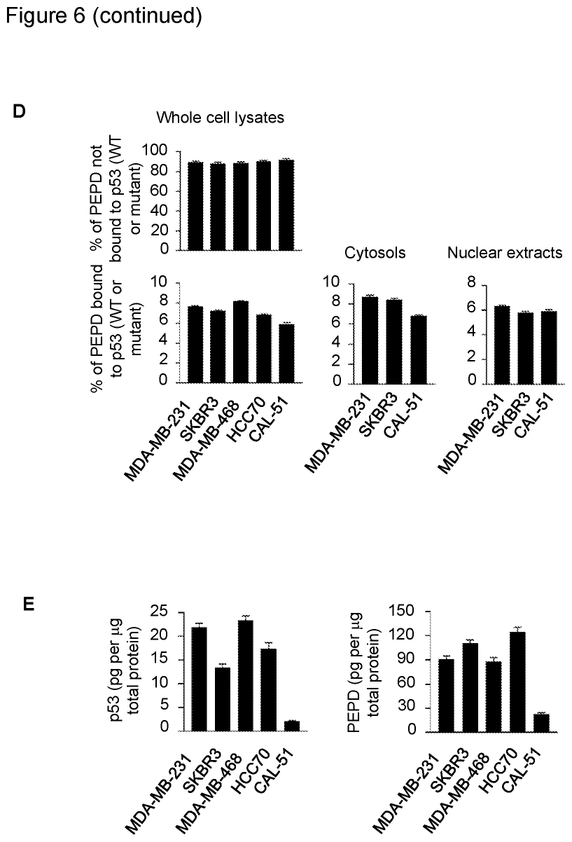

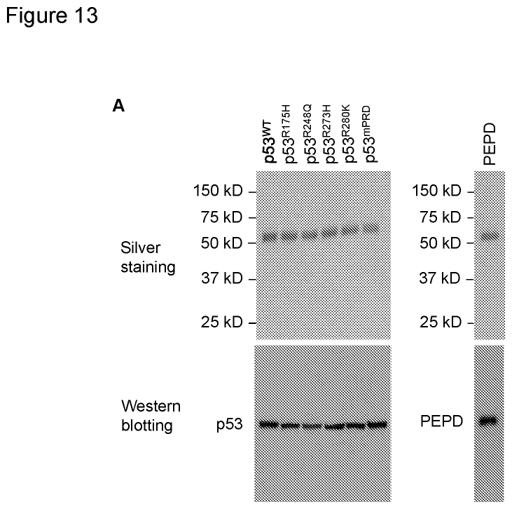

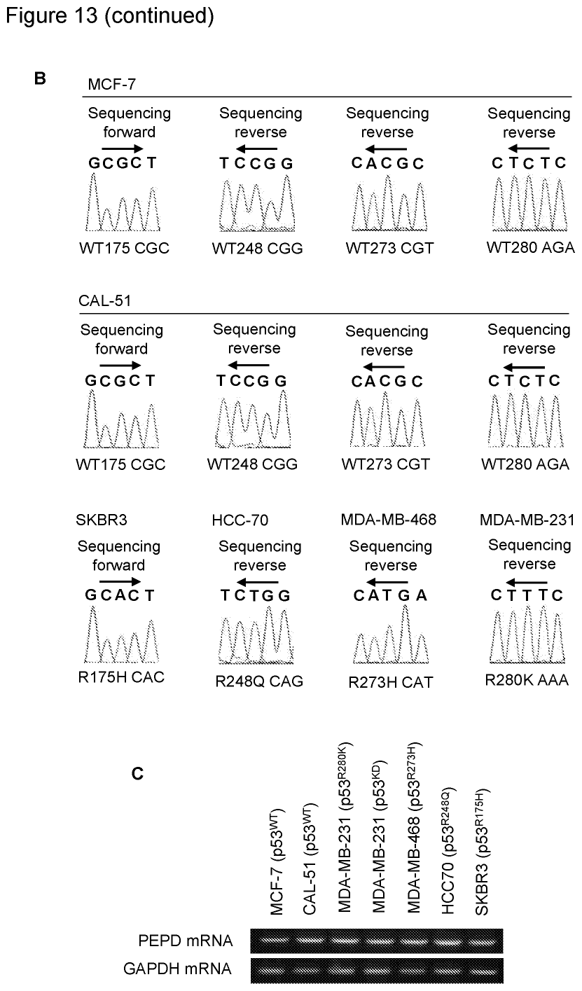

To obtain these results, we generated His-tagged recombinant human proteins in bacteria, including PEPD, p53 and their mutants, purified them by affinity chromatography, and confirmed their purity by gel electrophoresis and silver staining (see FIG. 13A). We measured PEPD binding to p53 and its mutants by ELISA. PEPD binds to p53^{WT}, p53^{R175H}, p53^{R248Q}, p53^{R273H} and p53^{R280K} with almost identical affinity, showing Kd of 145-185 nM (see FIG. 6A). We previously showed that PEPD does not bind to p53^{mPRD} in which all but one prolines in the PRD were changed to alanines (Yang et al., Nature Communications 2017, 8, 2052). We used p53^{mPRD} as a negative control to confirm that the ELISA specifically detects PEPD binding to PRD in p53.

We next measured PEPD binding to p53 mutants in cells. We screened seven cell lines, including SKBR3 (homozygous p53^{R175H}) HCC70 (homozygous p53^{R248Q})^{, MDA-MB-}468 (homozygous p53^{R273H}), MDA-MB-231 (homozygous p53^{R280K}), MDA-MB-231 (p53^{KO}), MCF-7 (p53^{WT}), and CAL-51 (p53^{WT}). They are human breast cancer cells, either HER2-positive (SKBR3), estrogen receptor-positive (MCF-7), or triple negative (all remaining cell lines). MDA-MB-231 (p53^{KO}) was generated from MDA-MB-231 by CRISPR-Cas9 knockout of TP53. MDA-MB-231 (p53^{R280K}) and MDA-MB-231 (p53^{KO}) constitute an isogenic pair of cells. We confirmed p53 genotype in each cell line by Sanger sequencing (FIG. 13B). We measured the expression of both p53 and PEPD in each cell line by Western blotting (WB). As expected, all the p53 mutants are overexpressed, compared to p53^{WT}, and p53 is absent in MDA-MB-231-p53^{K}G, but interestingly, PEPD is also overexpressed in cells carrying a p53 mutant and even in MDA-MB-231-p53^{KO} (FIG. 6B). An antibody recognizing both p53^{WT} and its mutants, DO-1, was used to detect p53 in WB.

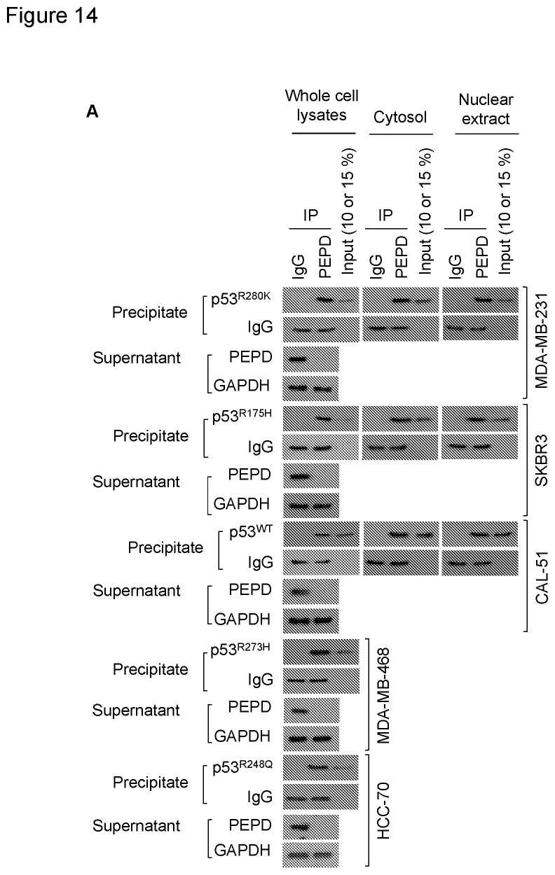

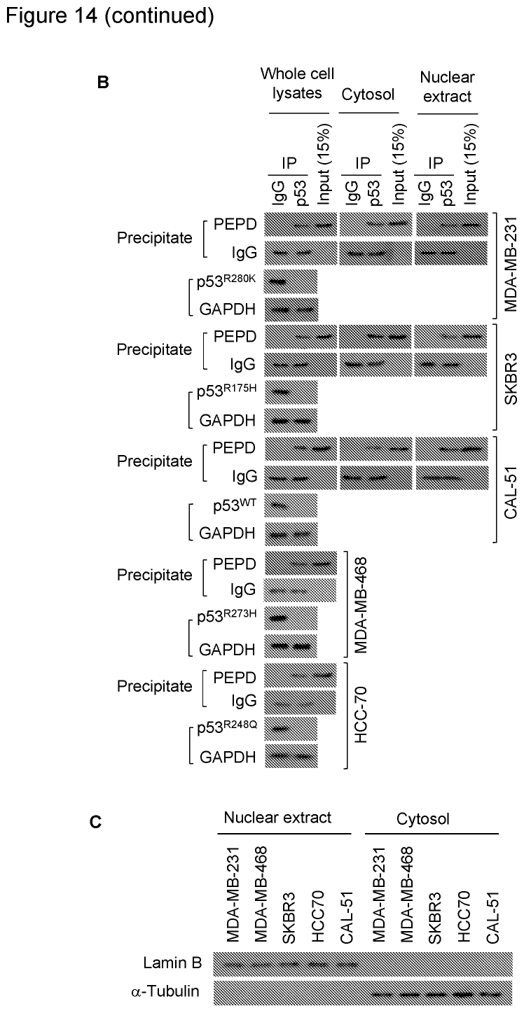

Based on these results, we measured PEPD binding to p53 mutants in whole cell lysates of SKBR3, MDA-MB-231, MDA-MB-468 and HCC70, and included CAL-51 for comparison to p53^{WT}. Because PEPD is present in both cytosol and nucleus we also measured PEPD binding to p53 in the cytosolic and nuclear fractions of MDA-MB-231, SKBR3 and CAL-51. PEPD or p53 in each sample was subjected to immunoprecipitation (TB) with excess antibody. Analysis of the supernatants by WB confirmed full pull-down of PEPD or p53 (FIG. 14A, 14B). The precipitates were analyzed by WB for p53 or PEPD along with an input (FIG. 14A, 14B), and the percentages of PEPD or p53 that co-precipitated with each other was calculated by comparing their band intensities with that of the inputs. Cross contamination between the cytosol and nuclear extract was ruled out by WB analysis of lamin B (a nuclear protein) and α-tubulin (a cytosolic protein) (FIG. 14C). For whole cell lysates, following IP, we also measured PEPD or p53 in the supernatants by ELISA to determine the percentages of PEPD or p53 molecules that did not bind to each other. The combined amount of PEPD or p53 detected in the precipitates and the corresponding supernatants accounted for 95-97% of PEPD and 93-96% of p53 in cells (FIG. 6C, 6D), which validates our experimental approach. Approximately 23% of p53w^{T} and 40-47% of the mutants bind to PEPD in the whole cell lysates, and the results are similar in the cytosol and nuclear extract (FIG. 6C). In contrast, only 6-9% of PEPD bind to p53WT and the mutants (FIG. 6D), even though cellular levels of PEPD are 3.1-8.7 fold higher than that of p53, as measured by ELISA (FIG. 6E).

### EXAMPLE 7

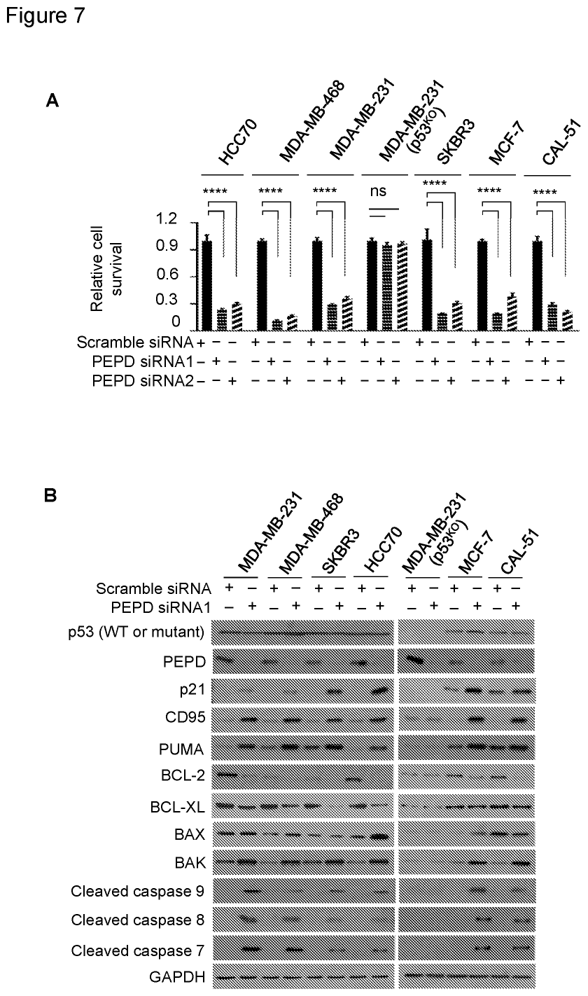

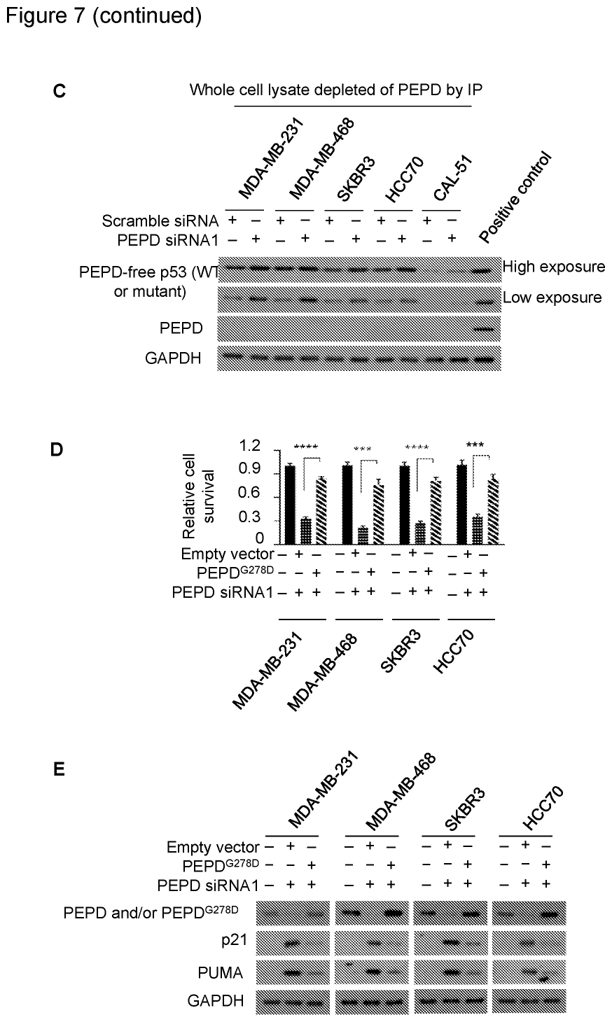

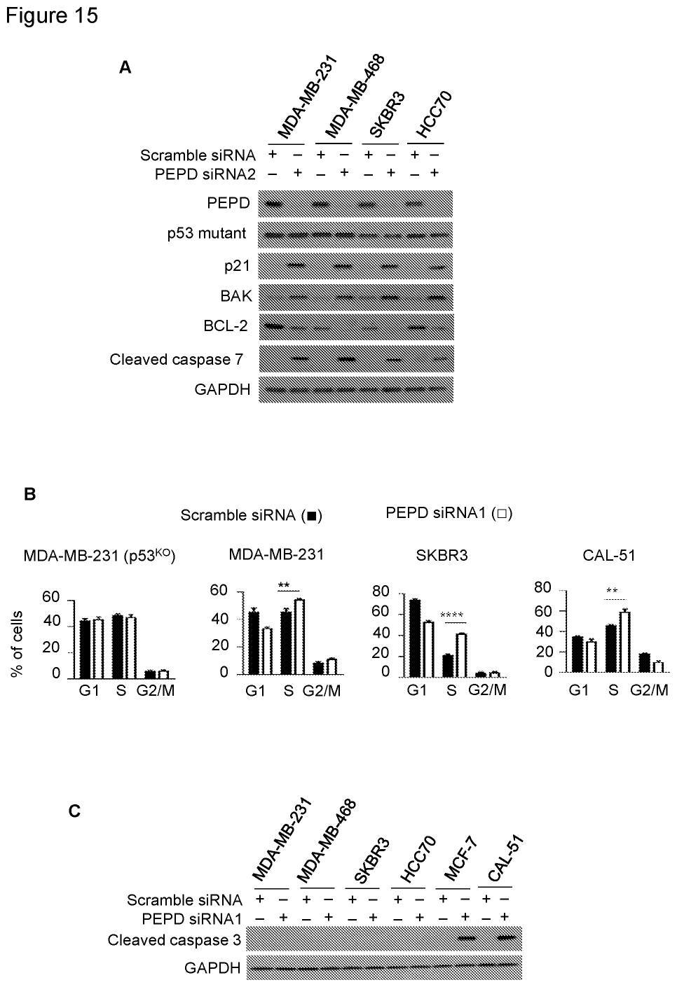

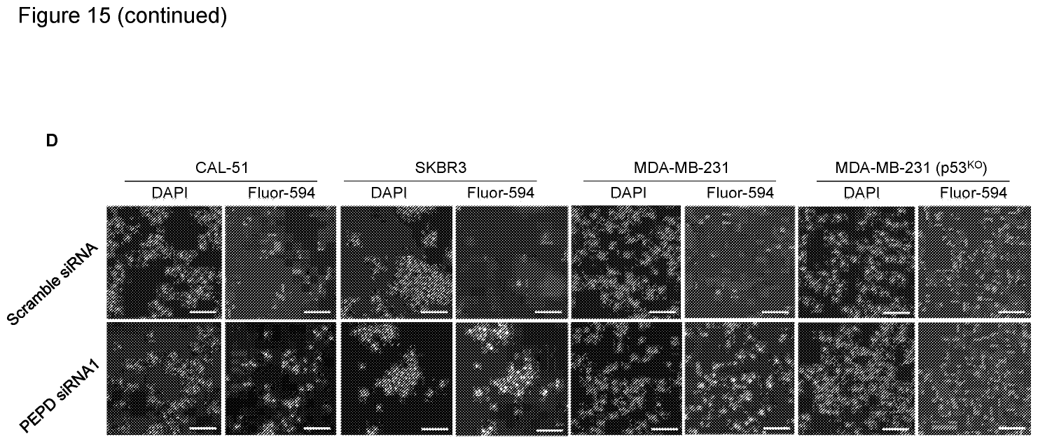

This Example demonstrates the effect of PEPD KD on cell survival and levels of key proteins, and the counter-effect of PEPD^{G278D}. The results are shown in FIG. 7. R175H, R248Q, R273H and R280K are among the most common cancer-associated p53 mutants and are well-known gain of function (GOF) mutants. Seven cell lines were used, including HCC70 (homozygous p53^{R248Q}), MDA-MB-468 (homozygous p53^{R273H}), SKBR3 (homozygous p53^{R175H}), MDA-MB-231 (homozygous p53^{R280K}) along with its p53-null isogenic counterpart (MDA-MB-231-p53^{-/-}), CAL-51 (p53^{WT}), and MCF-7 (p53^{WT}). All cell lines were from ATCC, except for MDA-MB-231-p53^{-/-}, which was generated from MDA-MB-231-p53R280K using CRISPR-Cas9 (Mukhopadhyay et al., J Natl Cancer Inst 2019, 111, djz051). We treated the cells with a control or PEPD siRNA (Origene) for 48-72 h. Two PEPD siRNAs, targeting exons 12 (siRNA2) and 15 (siRNAl), respectively, were used to rule out non-specific effects. Each PEPD siRNA caused cell death, killing 64-89% of cells carrying p53^{wT} or a mutant after 72 h treatment (FIG. 7A). However, neither siRNA was cytotoxic in MDA-MB-231-p53^{-/-} cells (FIG. 7A). In all cell lines carrying a p53 mutant or p53^{WT}, we detected strong regulation of p53 target genes upon PEPD KD, including induction of p21, CD95, PUMA, Bax and Bak, and downregulation of Bcl-2 and Bcl-xL, although regulation of some of the Bcl-2 family proteins is not uniform in the cell lines, but none of these changes occurred in the p53-null cells (FIG. 7B). There was no difference in the effects of the two PEPD siRNAs (FIG. 7A, 7B, 15A). Therefore, in later experiments, we focused on PEPD siRNA1. Because p21 inhibits cell cycle at G1/S phase, we also analyzed cell cycle by flow cytometry in MDA-MB-231, MDA-MB-231 (p53^{KO}), SKRB3, and CAL-51. PEPD siRNAl had no effect on cell cycle in MDA-MB-231 (p53^{KO}) but caused S phase arrest in the other cell lines (FIG. 15B). PEPD siRNA also caused activation of multiple caspases (caspase-9, -8, -7) in all the cell lines except MDA-MB-231 (p53^{KG}) (FIGS. 7B, 15A). These results show that PEPD KD not only activates p53^{WT} but also reactivates oncogenic p53 mutants.

Interestingly, PEPD KD activated caspase 3 only in p53^{WT} cells but not in any of the cell lines carrying a p53 mutant (FIG. 15C). Moreover, PEPD KD did not change total cellular levels of p53^{WT} and mutants (FIGS. 7B, 15A). However, by removing all PEPD from whole cell lysates using IP, including PEPD bound to p53, and measuring the supernatant for p53 by WB, we showed that PEPD KD frees p53^{WT} and mutants from PEPD (FIG. 7C).

We next investigated whether PEPD^{G278D}, an enzymatically inactive PEPD mutant that binds to PRD in p53 (Yang et al., Nature Communications 2017, 8, 2052) could neutralize the effects of PEPD siRNA on p53 mutants. Cells were transfected with a plasmid expressing PEPD^{G278D} and then treated with siRNA for 72 h. PEPD siRNA killed 65-79% of cells without PEPD^{G278D} but only 16-25% of cells with PEPD^{G278D} (FIG. 7D). PEPDG278D rescue is highly effective, since gene transfection efficiency is unlikely 100%. In cells expressing PEPD ^{G278D}, total levels of PEPD and PEPD^{G278D} did not decrease, and induction of p53^{WT} target proteins (p21 and PUMA) were largely abolished, despite PEPD siRNA treatment (FIG. 7E). These results provide further evidence that reactivation of p53 mutants by PEPD siRNA results from their separation from PEPD and indicate that the enzymatic activity of PEPD is not involved in regulating p53 mutants.

### EXAMPLE 8

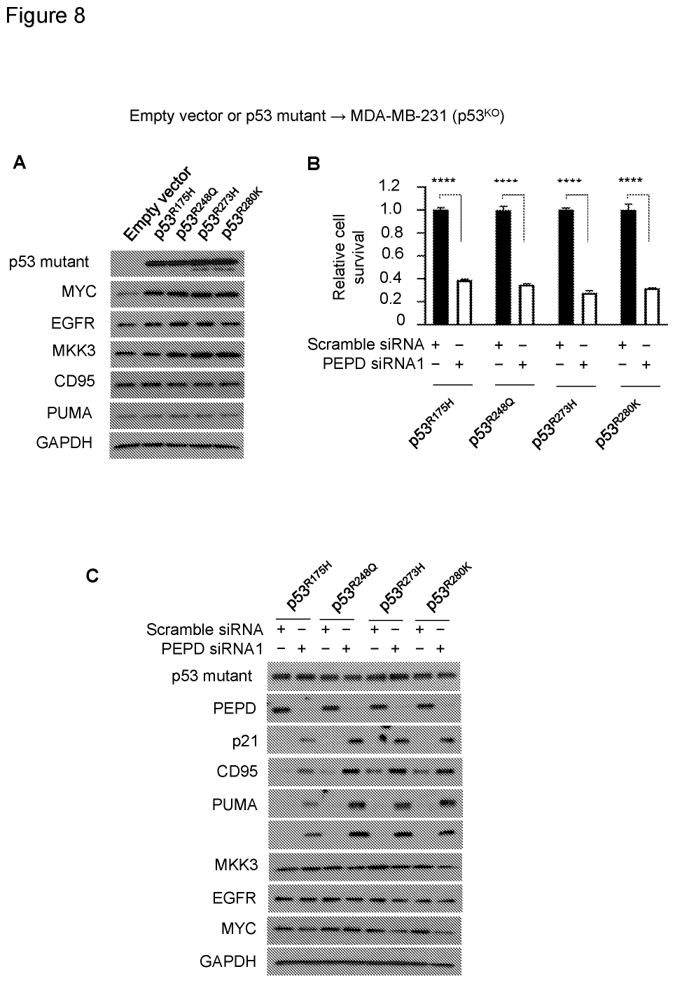

This Example demonstrates the effect of PEPD KD on p53 mutants in isogenic cells. It is important to note that more than 50% molecules of a p53 mutant are not bound to PEPD in cells (FIG. 6C). c-MYC (MYC), epidermal growth factor receptor (EGFR), and mitogen-activated protein kinase 3 (MKK3) are among the oncogenes upregulated by various p53 mutants. WB analysis showed that transfection of each of the four p53 mutants into MDA-MB-231 (p53^{KO}) cells induced MYC, and three of them also induced EGFR and MKK3, but no mutant induced CD95 and PUMA which are regulated by p53WT (FIG. 8A). However, if MDA-MB-231 (p53^{KO}) cells were transfected with a p53 mutant and 24 h later treated with PEPD siRNA, trypan blue assay showed marked inhibition of survival of cells expressing each mutant (FIG. 8B). WB analysis showed that PEPD siRNA causes no change in p53 level, marked PEPD KD, induction of p21, CD95 and PUMA, and activation of caspase 7, but no induction of MKK3, EGFR and MYC or even downregulation in some cases (FIG. 8C). These results along with those in FIG. 7 show that reactivation of p53 mutants by PEPD KD is not cell-context dependent and that reactivated mutant molecules dominate cell fate, although the unreactivated mutant molecules may still exert pro-oncogenic activities.

### EXAMPLE 9

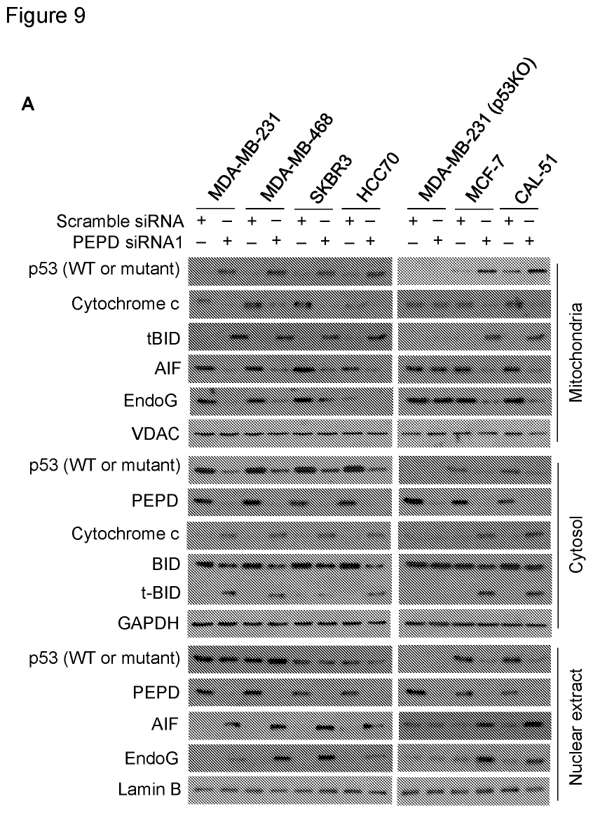

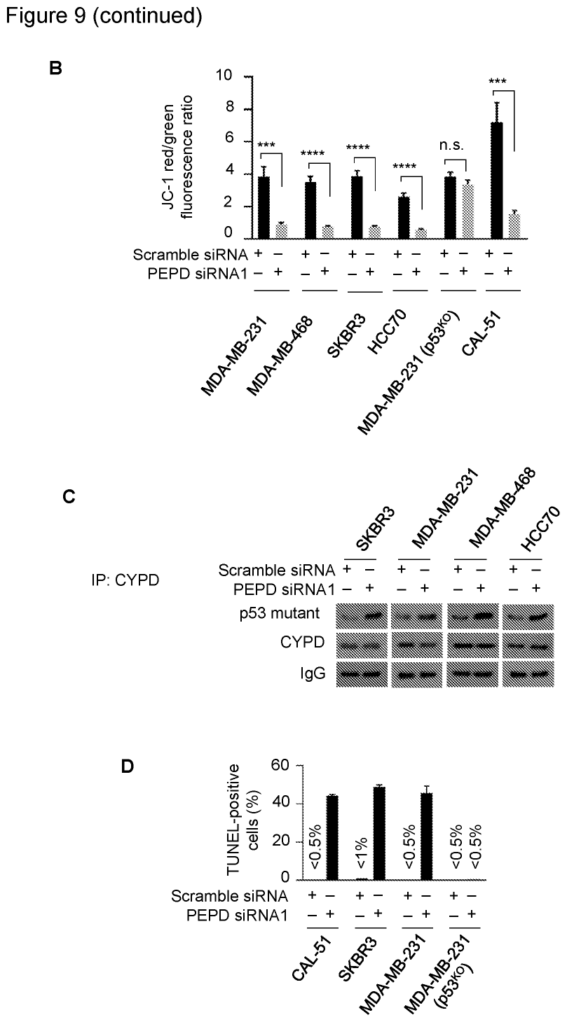

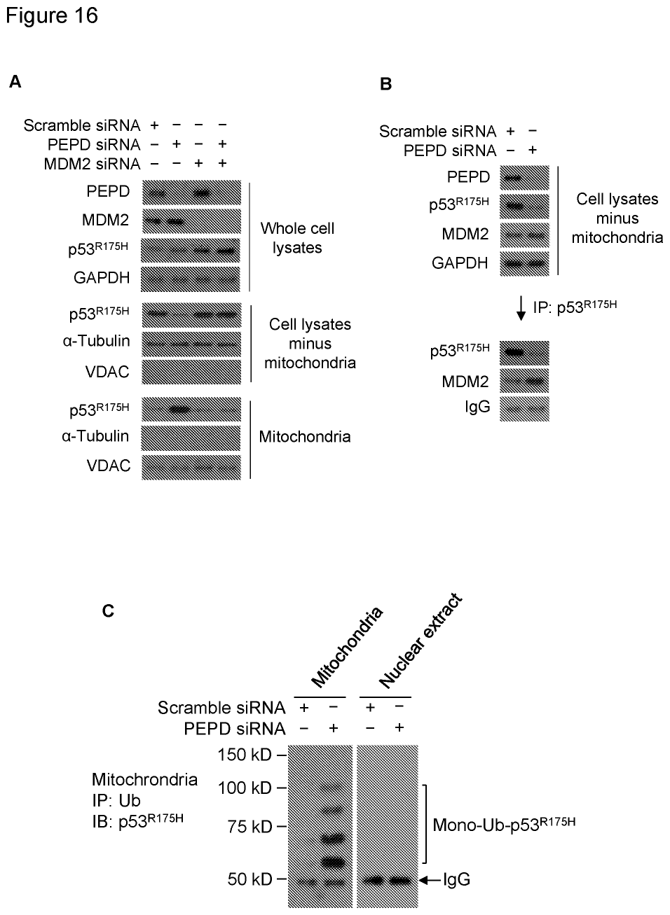

This Example demonstrates the effect of PEPD KD on transcription-independent tumor suppressor activities of p53 mutants. The results are demonstrated in FIG. 9, and are related to FIGS. 15 and 16. The results show that mitochondrial enrichment of p53, WT or mutant, was accompanied by mitochondrial enrichment of truncated BH3-interacting domain death agonist (tBID), mitochondrial loss of cytochrome c, apoptosis-inducing factor (AIF) and endonuclease G (EndoG), cytosolic increase in cytochrome c and tBID, cytosolic decrease in BID, and nuclear increase in AIF and EndoG (FIG. 9A). BID is known to be converted by caspase 8 to proapoptotic tBID which migrates to mitochondria. Nuclear translocation of AIF and EndoG and cytosolic translocation of cytochrome c from mitochondria are well established mechanisms of mitochondria-mediated apoptosis. However, although PEPD KD was pronounced in both cytosol and nucleus of MDA-MB-231 (p53^{KO}) cells, there was no subcellular redistribution of AIF, EndoG, cyto C, BID and tBID (FIG. 9A).

In line with the molecular changes described above, JC-1 fluorescence staining showed that PEPD KD by siRNA caused marked loss of mitochondria membrane potential (MMP) in cells carrying p53^{wT} or a mutant, and the extent of MMP lose was similar among cells with different p53 genotype, but PEPD KD did not significantly impact MMP in MDA-MB-231 (p53^{KO}) (FIG. 9B). It is well known that mitochondrial p53^{WT} causes MMP loss and cell death by binding to cyclophilin D (CYPD) to open mitochondrial permeability transition pore and by binding to BCL-2 and BCL-XL to neutralize their inhibitory effects on BAK and BAX. By co-IP and WB, we showed that PEPD siRNA markedly increases the binding of all p53 mutants to CYPD in mitochondria (FIG. 9C). However, because PEPD KD caused marked change in expression of BCL-2, BCL-XL, BAK and BAX (FIG. 7B), it was not feasible to assess the interactions of p53 mutants with the BCL-2 family proteins in mitochondria. We also performed TUNEL assay in select cell lines and showed that PEPD siRNA strongly induced apoptosis in cells carrying p53^{WT} or a mutant and but was inactive in p53^{KO} cells (FIG. 9D, 15D).

It is known that p53^{WT} translocation to mitochondria is driven by MDM2-mediated mono-ubiquitination. It is also known that MDM2 at low level of activity causes mono-ubiquitination of p53^{wT} but at high level of activity causes polyubiquitination and degradation of p53w^{T} Focusing on p53^{R175H} in SKBR3 cells, we showed that MDM2 KD by siRNA blocks PEPD KD-induced mitochondrial enrichment of p53^{R175H} (FIG. 16A), increases MDM2 binding to p53^{R175H} (FIG. 16B), and enriches mono-ubiquitinated p53^{R175H} in mitochondria (FIG. 16C). However, PEPD KD did not induce mono-ubiquitination of nuclear p53^{R175H} (FIG. 16C).

### EXAMPLE 10

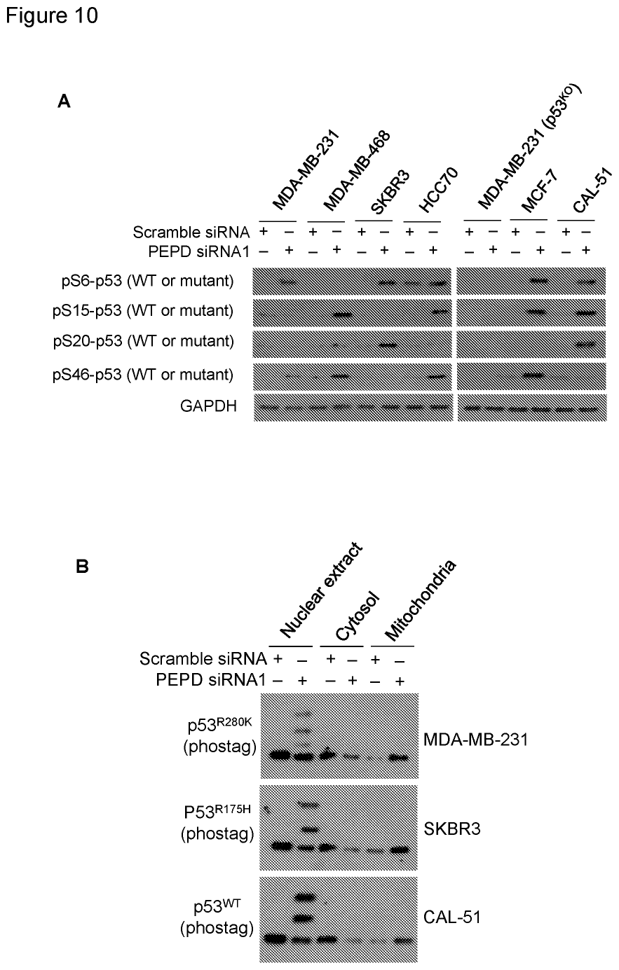

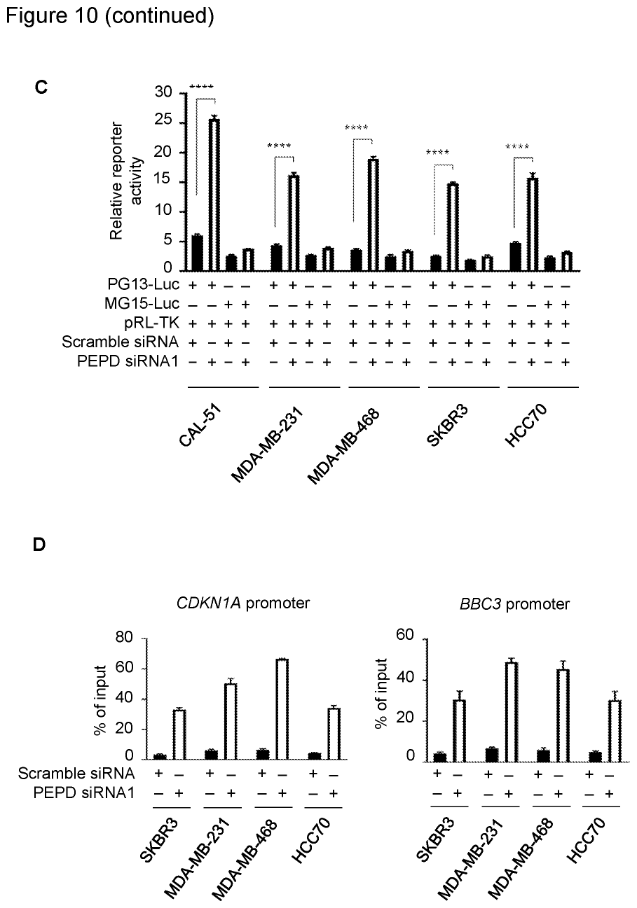

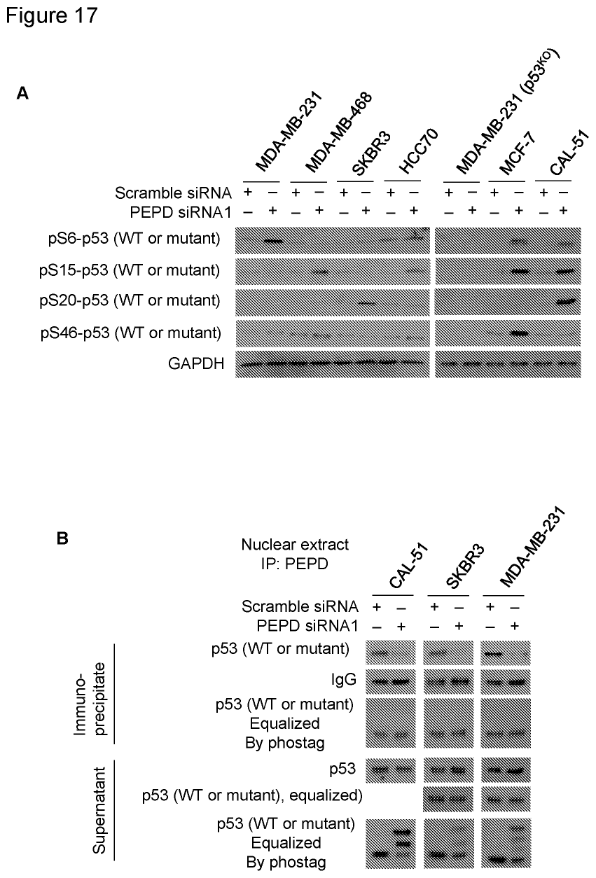

This Example demonstrates the effect of PEPD KD on phosphorylation and transcriptional activities of p53 mutants. The results are shown on FIG. 10 and FIG. 17. PEPD KD apparently also reactivates the transcriptional functions of p53 mutants (FIG. 7B). Because phosphorylation is a well-known crucial step in activation of p53^{WT} transcriptional activity, we measured four phosphorylation sites in the transactivation domains (S6, S15, S20, S46) of p53^{WT} and mutants by WB. PEPD KD induced phosphorylation of p53^{WT} and all the mutants, but the phosphorylation sites varied among them (FIGS. 10A, 17A). The variability is at least partly related to cell context, because phosphorylation sites in p53^{wT} also differ between MCF-7 and CAL-51 (FIGS. 10A, 17A). Because there was no shared phosphorylation site, we next used phostag WB, which detects overall phosphorylation, to determine the cellular location of phosphor-p53. We focused on p53^{R280K} (MDA-MB-231), p53^{R175H} (SKBR3), and p53^{WT} (CAL-51). Phospho-p53 (WT and mutants) induced by PEPD KD occurred in the nucleus but not in the cytosol or mitochondria (FIG. 10B). We removed all PEPD from the nuclear extract by IP and measured phosphor-p53 in the precipitate and the supernatant, but detected phospho-p53 (WT and mutants) only in the supernatant (FIG. 17B), indicating that p53 is phosphorylated after leaving PEPD. Next, cells were transfected with PG13-luc or MG15-luc along with pRL-TK for control of transfection efficiency and then treated with siRNA for 48 h. PEPD siRNA induced reporter expression from PG13-luc by 4.2 fold in CAL-51 cells (p53^{WT}) and 3.3-5.6 fold in cells carrying a p53 mutant (FIG. 10C). In contrast, PEPD siRNA had negligible effect on reporter expression from MG15-luc in all the cells. Using ChIP-qPCR, we further showed that in cells treated with PEPD siRNA for 48 h, binding of each p53 mutant to the p53^{WT} binding site in the promoter of CDKAT1A gene which encodes p21 increased 7.6-10.2 fold and in the promoter of BBC3 gene which encodes PUMA increased 5.9-7.6 fold (FIG. 10D).

Collectively, the above results, together with other results shown before, reveal that p53 mutants, once freed from PEPD by PEPD KD, are phosphorylated and regulate p53^{WT} target genes by binding to p53^{WT} binding sites in their promoters. The reactivated transcriptional activities of p53 mutants are almost indistinguishable from that of activated p₅₃^{WT}.

### EXAMPLE 11

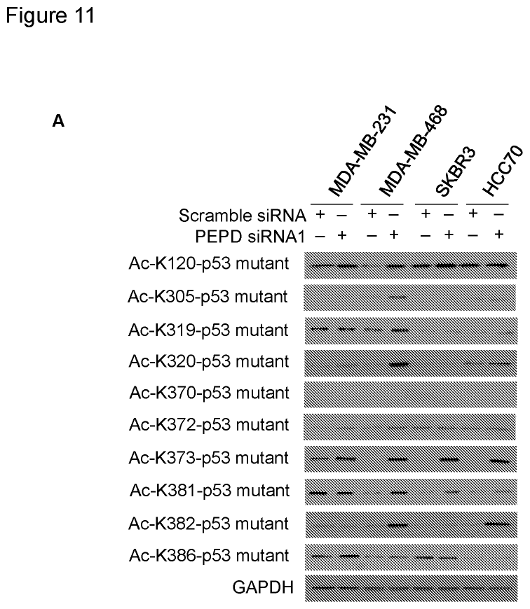

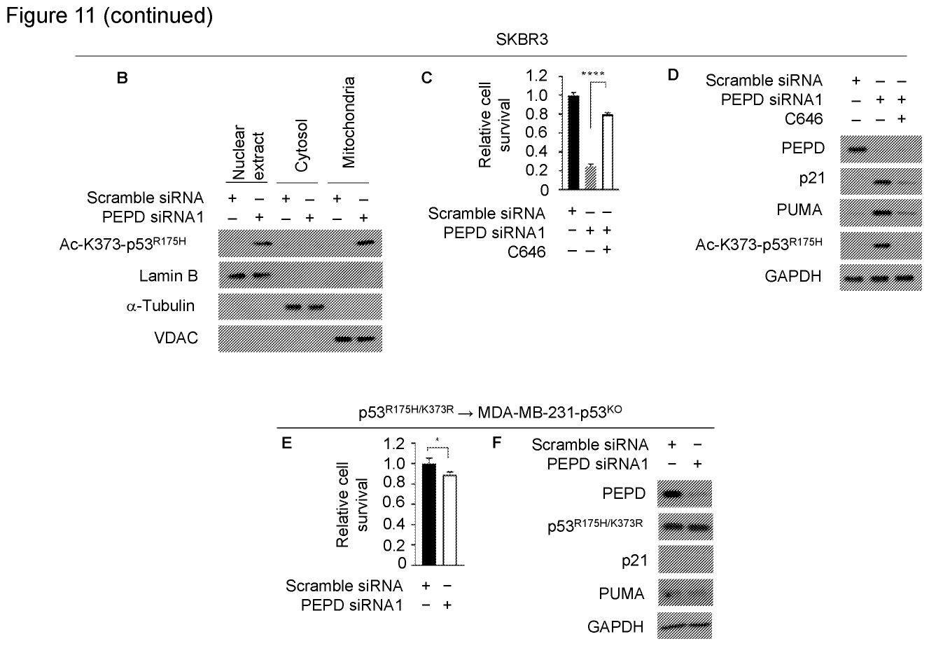

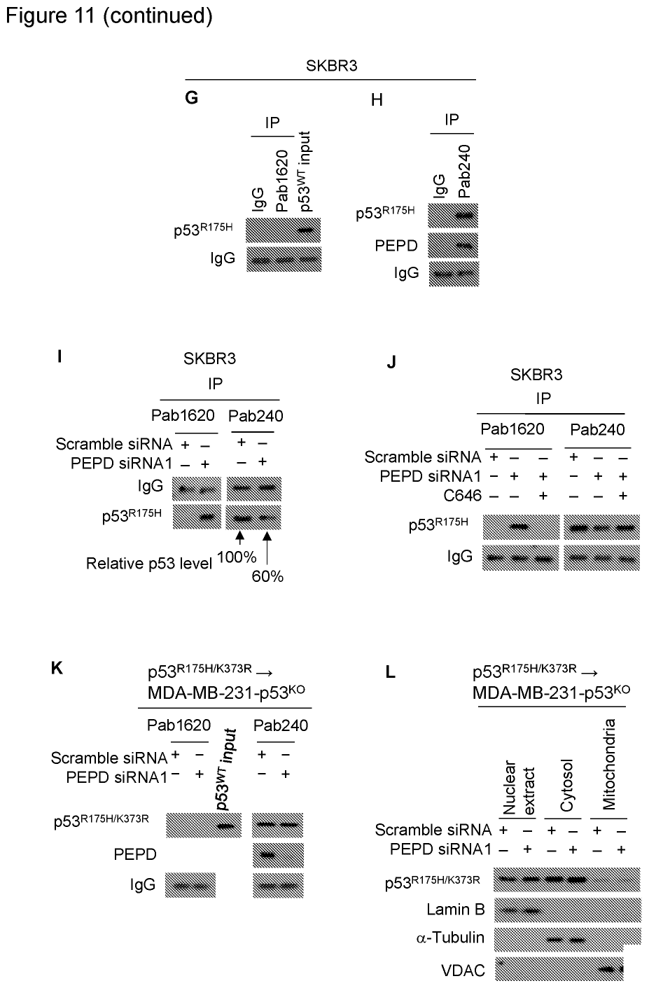

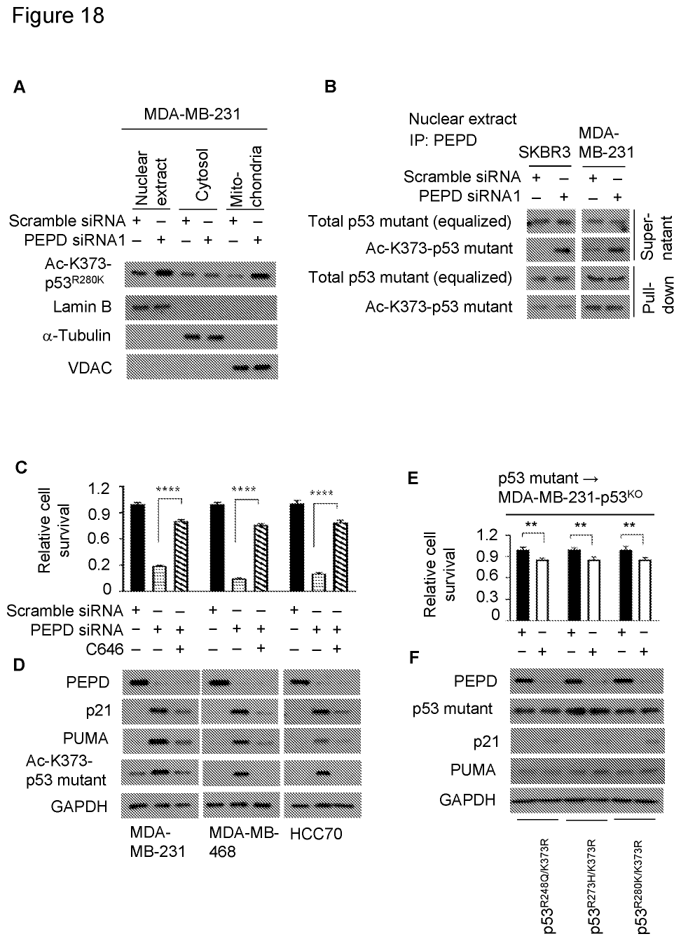

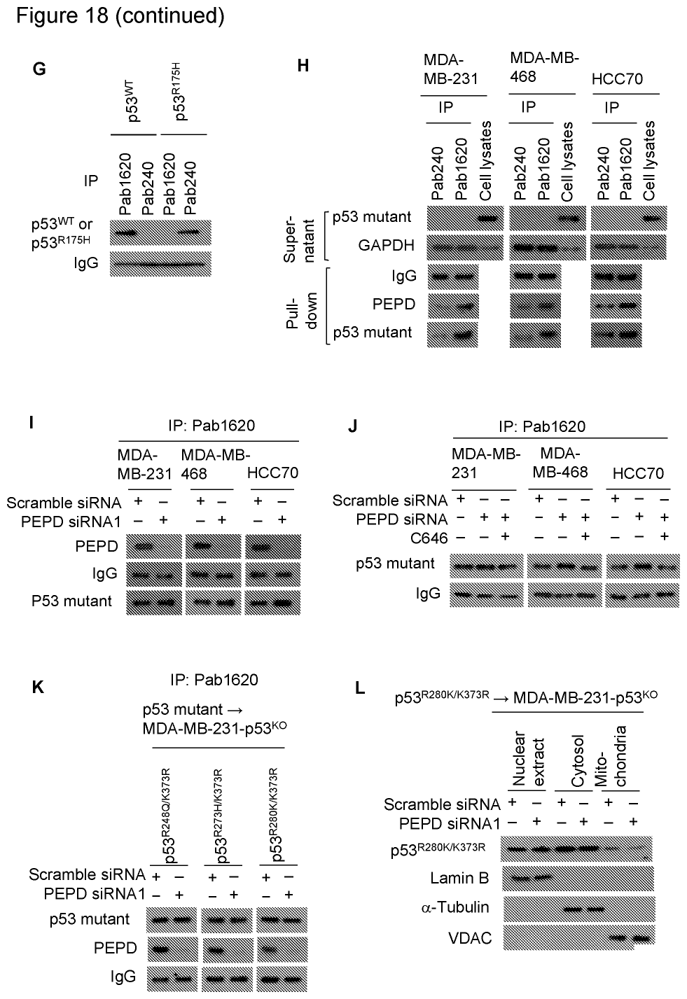

This Example shows the effect of PEPD KD on refolding and reactivation of p53 mutants, and the role of K373 acetylation. The results are shown on FIG. 11 and FIG. 18. The results indicate that acetylation at K373 is essential for reactivation of p53 mutants, and p53 mutants refold once separated from PEPD, which is driven byK373 acetylation. It is well known that acetylation of p53^{wT} is critical for its activation. We measured the effect of PEPD KD on acetylation at various lysine residues of p53 mutants by WB. PEPD KD by siRNA induced acetylation at various sites, but only K373 acetylation is shared by all the mutants, and there were no K370 acetylation and little change in K372 acetylation (FIG. 11A). Focusing on SKBR3 cells (^{p53R175H}) and MDA-MB-231 cells (p53^{R280K}) we showed that K373 acetylation occurs to the mutants in the nucleus and mitochondria but not cytosol (FIGS. 11B, 18A). We next asked whether K373 acetylation occurs before or after the mutants leave PEPD. Because PEPD is not present in the mitochondria, we analyzed nuclear extracts. We removed all PEPD from the nuclear extract using IP and measured Ac-K373-p53 mutants in the precipitate and supernatant by WB. Ac-K373-p53 mutants, resulting from PEPD KD, were present only in the supernatant (FIG. 18B). Thus, K373 acetylation occurs after the mutants leave PEPD. C646 is known to inhibit p300/CBP that acetylates p53. C464 prevented PEPD siRNA from causing death of all the cell lines carrying a p53 mutant (FIGS. 11C, 18C). C464 also blocked K373 acetylation of p53 mutants and induction of p53^{WT} target proteins, including p21 and PUMA, in all the cell lines, despite profound PEPD KD (FIGS. 11D, 18D). To further clarify the role of K373 acetylation in reactivation of p53 mutants, we introduced K373R mutation to each p53 mutant and expressed the mutant in MDA-MB-231 (p53^{KO}) cells, followed by PEPD KD by siRNA for 96 h. K373R mutation rendered each p53 mutant almost totally non-responsive to PEPD KD as assessed by cell survival and induction of p21 and PUMA (FIGS. 11E, 11F, 18E, 18F). Thus, K373 acetylation is the key molecular switch for reactivation of p53 mutants by PEPD KD. We wondered whether PEPD binds to p53 mutants to scaffold them back to wild-type conformation. Antibodies Pab1620 and Pab240 are widely used to detect p53 in “wild-type” and “denatured” conformations, respectively, although they do not inform the precise folding state of the protein. We confirmed that Pab1620 and Pab240 specifically detect the “wild-type” and “denatured” conformation respectively (FIG. 18G). p53^{R175H} is known to exist entirely in “denatured” confirmation. Indeed, IP followed by WB showed that p53^{R175H} in SKBR3 cells does not bind to Pab1620 but binds to Pab240 (FIGS. 11G, 11H). PEPD was pulled down with p53^{R175H} by Pab240 (FIG. 11H), indicating that p53^{R175H} remains in “denatured” conformation after binding to PEPD. About 40% of p53^{R1751-1} molecules switched from “denatured” to “wild-type” conformation after PEPD siRNA treatment for 48 h (FIG. 11I). The refolding of about 40% p53^{R175H} closely matches the percentage of p53^{R175H} initially binding to PEPD in SKBR3 (FIG. 6C), indicating that all p53^{R175H} molecules refold after leaving PEPD. We also examined p53^{R248Q} p53^{R273H} and p53^{R280K}. Whole cell lysates were subjected to IP by Pab240 or Pab1620, and analysis of the supernatants by WB confirmed complete pull down of each p53 mutant (FIG. 18H). Analysis of precipitates by WB showed that each mutant exists mainly in “wild-type” conformation, with the rest in “denatured” conformation (FIG. 18H), which is consistent with previous reports. PEPD was pulled down with each p53 mutant by both Pab1620 and Pab240, but mainly by Pab1620 (FIG. 18H). Thus, PEPD binds to p53 mutants regardless of their conformation and does not induce refolding of the mutants. However, PEPD KD increased the levels of the three mutants in “wild-type” conformation (FIG. 181), and the relatively small increases closely match the relatively small pool of the mutant molecules existing initially in “denatured” conformation. Notably, PEPD KD does not change the total levels of p53 mutants (FIG. 7B). Thus, mutant molecules that are in “denatured” conformation may all change to “wild-type” conformation after leaving PEPD. Collectively, our results show that PEPD binding to p53 mutants does not change their conformation but the mutants refold after leaving PEPD.

C646 blocked PEPD KD-induce conformation change of all four p53 mutants from “denatured” to “wild-type” (FIGS. 11J, 18J). We next expressed p53^{R175H/K373R}, p53^{R248Q/K373R}, p53^{R273H/K373R} or p53^{R280K/K373R} in MDA-MB-231 (p53^{KO}) cells, which were then treated with scramble or PEPD siRNA. K373R mutation did not change binding of each p53 mutant to PEPD but completely blocked conformation change of the mutants induced by PEPD KD (FIGS. 11K, 18K). Therefore, K373 acetylation, which occurs after p53 mutants leave PEPD, drives their refolding. Moreover, using p53^{R175H/K373R} and p53^{R280K/K373R} we showed that neither double mutant, when expressed in MDA-MB-231 (p53^{KO}) cells, was enriched in mitochondria when the cells were treated by PEPD siRNA (FIGS. 11L, 18L). Collectively, K373 acetylation is fundamental to reactivation of both transcription-dependent and -independent tumor suppressor functions of p53 mutants by PEPD KD.

### EXAMPLE 12

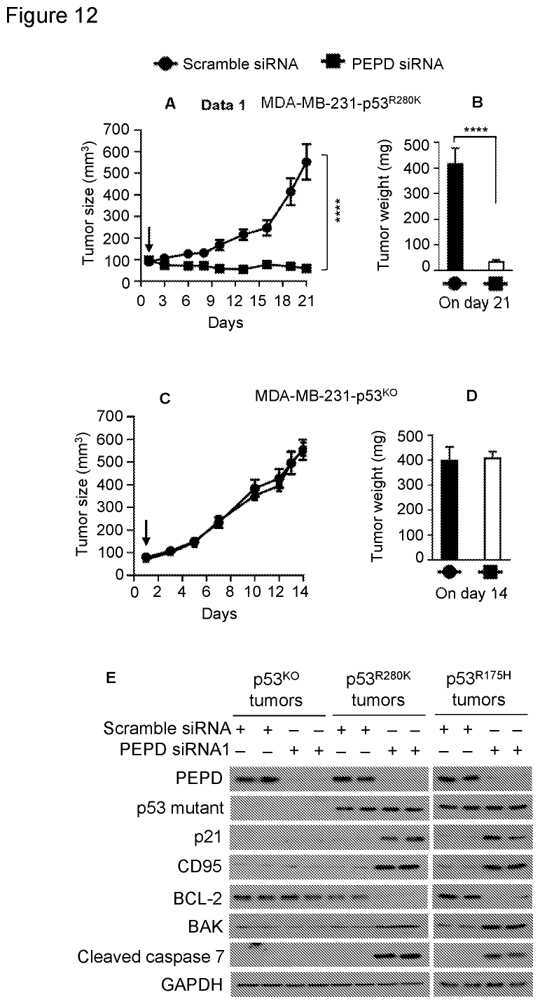

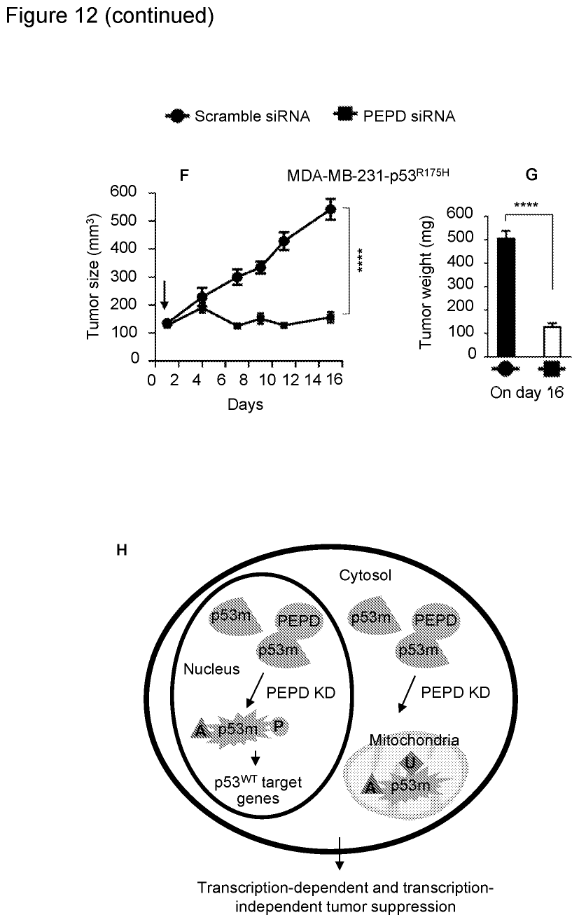

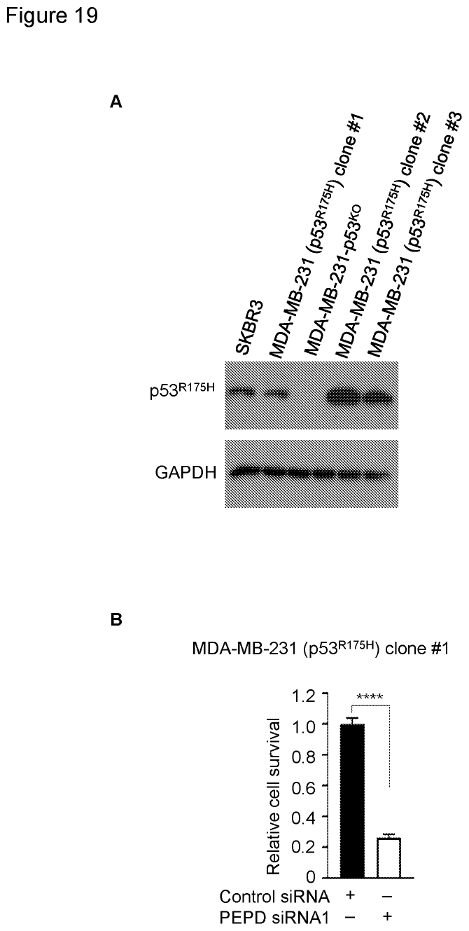

This Example shows the in vivo effect of PEPD KD on the growth of and expression of key proteins in isogenic tumors with or without expression of a p53 mutant. The results are shown in FIG. 12, and are related to FIG. 19. The data demonstrate that reactivated p53 mutants induced PEPD KD via intra-tumoral injection of PEPD siRNA strongly inhibit tumor growth in relevant animal models with orthotopic breast tumors generated from MDA-MB-231-p53^{R280K}, MDA-MB-231-p53KO or MDA-MB-231-p53^{R17514} cells.

We first compared the effects of PEPD KD on isogenic orthotopic tumors differing only in p53. We inoculated MDA-MB-231 cells (p53^{R280K}) or MDA-MB-231 (p53^{KO}) cells to the mammary fat pads of female SCID mice. Once tumors reached about 100 mm³, intratumor injection of scramble or PEPD siRNA (10 pmol) was given once every three days. The experiment was stopped when average tumor size in the control mice reached approximately 550 mm³, in order to minimize impediment to siRNA distribution into tumor tissue. MDA-MB-231 (p53^{KO}) tumors and MDA-MB-231 (p53^{R280K}) tumors were collected from the mice one and two days after the final treatment, respectively. We detected no adverse effect of the siRNA on the mice. Both types of tumors grew rapidly on scramble siRNA. However, PEPD siRNA strongly inhibited the growth of MDA-MB-231 (p53^{R280K}) tumors, and at the end of the experiment, average tumor size and tumor weight were only 10.6% and 8.6% of that in the control group respectively (FIG. 12A, 12B). In contrast, PEPD siRNA had no effect on the growth of MDA-MB-231(p53^{KO} tumors (FIG. 12C, 12D). Analysis of representative tumor samples by WB showed PEPD levels are similar between the two types of tumors and PEPD siRNA caused pronounced PEPD KD in both tumors. In MDA-MB-231 (p53^{R280K}) tumors, PEPD siRNA did not change total p53 level but strongly upregulated p21, CD95 and BAK, downregulated BCL-2, and activated caspase 7 (FIG. 12E), which closely resemble the changes detected in vitro (FIG. 7B). In contrast, in MDA-MD-231 (p53^{KO} tumors, PEPD siRNA had no effect on p21, CD95, BCL-2, BAK and caspase 7 (FIG. 12E).

Because p53^{R280K} is a contact mutant, we also investigated p53^{R175H}, which is a conformation mutant and is completely “denatured”. SKRB3 cells carry p53^{R175H} but failed to generate tumors in vivo, regardless of mouse strain (SCID, NOD SCID, or nude) or route of inoculation (subcutaneous or orthotopic). Since p53^{R17H} expressed transiently in MDA-MB-231(p53^{KO} cells was reactivated by PEPD KD (FIG. 8B, 8C), we used these cells to generate MDA-MB-231 cells stably expressing p53^{R175H}. Several p53^{R175H}-expressing clones were screened by WB, and clone #1, whose p53^{R175} level is similar to that of SKBR3 (FIG. 19A), was chosen. The advantage of MDA-MB-231 (p53^{R175H}) is that it is isogenic to MDA-MB-231 (p53^{KO} which does not response to PEPD KD in vitro and in vivo. We first confirmed that PEPD siRNA strongly inhibits the growth of MDA-MB-231 (p53 ^{R175H}) cells in vitro (FIG. 19B). We next inoculated MDA-MB-231 (p53^{R175H}) cells to the mammary fat pads of female SCID mice, and tumors grew rapidly. When tumors reached about 130 mm³, intratumor injection of scramble or PEPD siRNA (10 pmol) was started, which was given once every three days. The experiment was stopped once average tumor size in the control mice reached about 550 mm³. Tumors were collected from the mice two day after the final treatment. Again, there was no adverse effect of the siRNA on the mice. Whereas tumors grew rapidly on scramble siRNA, PEPD siRNA strongly inhibited tumor growth, and at the end of the experiment, average tumor size and tumor weight were only 28.9% and 25.9% of that in the control group, respectively (FIG. 12F, 12G). Analysis of representative tumor samples by WB showed that PEPD siRNA caused pronounced PEPD KD, did not change p53 level, but strongly upregulated p21, CD95 and BAK, downregulated BCL-2, and activated caspase 7 (FIG. 12E), consistent with the reactivation of p53^{R175H}.

Notably, in a pair of isogenic tumors in mice derived from human colon cancer HCT116 cells (p53^{WT}) and HCT116 cells (p53^{KO}), PEPD KD by intratumor injection of siRNA inhibited the growth of p53^{WT} tumors by 79% with strong activation of p53 target genes but had no effect on p53^{KO} tumors (Yang et al., Nature Communications 2017, 8, 2052). Thus, the in vivo tumor suppressing activities of p53^{R175H} and p53^{R280K} reactivated by PEPD KD are similar to that of p53^{WT} activated by PEPD KD.

### EXAMPLE 13

This Example provides a characterization of PEPD, a PEPD mutant, p53^{WT}, p53 mutants, and cell lines. The results are shown on FIG. 13 and are related to FIG. 6 and FIG. 7.

### EXAMPLE 14

This Example demonstrates binding of PEPD to p53^{WT} and its mutants in cells. Results are shown on FIG. 14 and are related to FIG. 6.

### EXAMPLE 15

This Example demonstrates the effect of PEPD KD on levels of p53 and other proteins, cell cycle progression, and apoptosis. Results are shown in FIG. 15 and are related to FIG. 7 and FIG. 9.

### EXAMPLE 16

This Example demonstrates the role of MDM2 in mitochondrial enrichment of p53^{R17}′ in SKBR3 cells. The results are shown in FIG. 16 and are related to FIG. 9.

### EXAMPLE 17

This Example demonstrates the effect of PEPD KD on phosphorylation of p53^{WT} and p53 mutants. The results are shown in FIG. 17 and are related to FIG. 10.

### EXAMPLE 18

This Example demonstrates the effect of PEPD KD on refolding of p53 mutants, and the role of K373 acetylation in refolding and reactivation of p53 mutants. Results are shown in FIG. 18 and are related to FIG. 11.

### EXAMPLE 19

This Example provides a characterization of MDA-MB-231 cells stably expressing p53^{R175H}. The results are shown in FIG. 19 and are related to FIG. 12.

The Examples above are intended to illustrate certain embodiments but not limit the scope of this disclosure.

1. A method for inhibiting the growth of cancer, the method comprising administering to cancer cells that express a p53 mutant that promotes cancer growth an RNAi agent that inhibits expression of prolidase (PEPD) or an agent that disrupts an association of a p53 mutant with PEPD.

2. The method of claim 1, wherein inhibiting the expression of PEPD comprises inhibiting formation of a complex comprising the p53 mutant and the PEPD.

3. The method of claim 1, wherein the RNAi agent comprises an siRNA.

4. The method of claim 1, wherein the p53 mutant comprises a loss of function p53 mutant, dominant negative p53 mutant, or a gain of function p53 mutant.

5. The method of claim 4, wherein the p53 mutant comprises a loss of function p53 mutant, dominant negative p53 mutant, or a gain of function p53 mutant, wherein optionally said p53 mutant is selected from a mutation in the p53 protein that is a change of at least one p53 amino acid that is amino acid R175, R248, R273, R280, or E285.

6. The method of claim 5, wherein the administration of the RNAi agent results in a p53 mutant that was previously in a complex with PEPD being freed from contact with the PEPD.

7. The method of claim 6, wherein the p53 mutant that is freed from contact with the PEPD participates in killing of the cancer cells.

8. The method of claim 1, wherein the cancer cells are present in an individual who has been diagnosed with cancer comprising cancer cells that express a p53 mutant.

9. The method of claim 4, wherein the cancer cells are present in an individual who has been diagnosed with cancer comprising cancer cells that express a p53 mutant.

10. The method of claim 5, wherein the cancer cells are present in an individual who has been diagnosed with cancer comprising cancer cells that express a p53 mutant.

11. The method of claim 6, wherein the cancer cells are present in an individual who has been diagnosed with cancer comprising cancer cells that express a p53 mutant.

12. The method of claim 7, wherein the cancer cells are present in an individual who has been diagnosed with cancer comprising cancer cells that express a p53 mutant.

13. The method of claim 3, wherein the cancer cells are present in an individual who has been diagnosed with cancer comprising cancer cells that express a p53 mutant.

14. The method of claim 1, further comprising selecting the individual to receive treatment with the RNAi agent based on a determination that the individual has a cancer that expresses the p53 mutant.

15. The method of claim 14, wherein the cancer that expresses the p53 mutant comprises a loss of function p53 mutant, dominant negative p53 mutant, or a gain of function p53 mutant.

16. The method of claim 15, wherein the p53 mutant comprises a mutation in the p53 protein selected from a change of at least one p53 amino acid that is amino acid R175, R248, R273, R280, or E285.
